SEQUENCE LISTING (30 sequences)

SEQ ID NO: 1 - DNA, 1686 bp, Thermus antranikianii
gtggaggggtttgaactccactacatcccggaagtaggccccggcatgggggagcttttggacctcctcatgcgccagcccgtcctgggggtggacctggaaaccacggggcttgacccccacacctcgaggccccggctcctctccctggccatgccgggggcggtggtcgtctttgacctgttcggcgttccccttgaagtcttctaccccctcttctcccgggaggaggggcccttgctggtgggccacaacctgaagtttgacctcctcttcctcctcaaggccggggtgtggcgggctagcggcaagaggctttgggacaccggactggcccaccaggtgcttcacgcccaagcccgcatgcccgccctcaaggacttagcgccggggctagacaagaccctgcagacctcggactggggtggccccctctcctcggaacaggtggcctacgccgcccttgacgcggccgtgcctctggtcctgtaccgggagcagagggaacgggccagaaccctcaggcttgagaaggtcctggaggtggagcgccgcgcccttcccgccgtggcgtggatggagcttcggggggtgcccttcgccccggaactctgggaggaggccgccagggaagcggaacgggaggcggaagccctacgcggggaactccccttcggggtgaactggaacagccccgcccaggtgctggcctacctgaagggggagggtttggatctccccgacacccgggaggacaccctggccggctaccgggagcaccccctggtggccaagctcctccggtaccgggaagcggccaagcgggtgagcacctacgggaaggagtgggccaagcacctgaacccggccacgggacgcatacacccttcctggcaacagataggggcggaaacgggccgcatggcttgccggaagcccaaccttcagcaggtgccccgggatcccgccctgagaagggccttccggcctaaggaggggcgggtcatgctcaaggccgacttctcccagattgagctacggattgccgccgccatagccaaggaggggcggatgctcagggcgttccgggaggggaaggacctccacgccctcaccgccagcctggtcctggggaagcccctggaagaggtgggcaaggaggaccggcaactggccaaggcgctgaacttcgggcttctctacgggctgggggcggaagggctgaggaggtacgccctcaccgcctacggggtgaagctcaccctcgaggaggcccagaagcttcgggacgcgttcttccgggcttaccccgccctgaagcgctggcaccggtcccagcctgagggggaggtggtggtgaggaccctcttgggccggaggaggaccacggaccgctacacggaaaagctcaacaccccggtacagggaaccggggcggacgggctcaagatggccctggccctcctctgggagaaccggggcctactctggggagccttccccgtcctggcggtgcatgacgaggtggtgctggaggcccccgaggagggggccaaggagtacctggaaaccctcaccgccctcatgcgccaggggatggaggaggtgcttgggggcgcggtgcccgtggaggtggaaggaggcatctaccgggactggggggccacgccgtgggaggaggcatga

SEQ ID NO: 2 - DNA, 1686 bp, Thermus brockianus
atggaggggtttgaactccactacatcccggaagtaggccccggcatgggggagcttttggacctcctcatgcgccagcccgtcctgggggtggacctggaaaccacggggcttgacccccacaccgcacgccccaggctcctctctctggccggggagcggtttgccgtggtggtggacctcttccgggtgccccttgaagtcttccgccccctcttctcctgggaggaggggccccttttggtggggcacaacctcaagtttgacctcctcttcctcctcaaggccggggtgtggcggggaagcggcagaaggctttgggacaccggactggcccaccaggtgcttcacgcccaagcccgcatgcccgccctcaaggacttagcgccggggctagacaagaccctgcagacctcggactggggtggccccctctcctcggaacaggtggcctacgccggtcttgacgcggtggtgcccctctccctctacggggagcagaagaagcgggcccgggccatggggcttgagaaggtccttgaggtggagcaccgcgccctccccgccgtggcgtggatggagcttcggggggtgcccttcgccccggaactctgggaggaggccgccagggaagcggaacgggaggcgaaagccctacgcgcggaactccccttcggggtgaactggaacagccccgcccaggtgctggcctacctgaagggggaggggctggaccttcccgacacccgggaggacaccctggccggctaccgggagcaccccctggtggccaagctcctccggtaccgggaagcggccaagcgggtgagcacctacgggaaggagtgggccaagcacctgaacccggccacgggacgcatacacccttcctggcaacagataggggcggaaacgggccgcatggcgtgccgcaagcccaacctccagcaggtgccccgggaccccgccctgcgaagggcgttccggccccccgagggcaaggtgctcctcaaggccgacttctcccagattgaactgcggattgccgccgccatagcccgggaagggcggatgctccaagcgttccgggaggggaaggaccttcacgccctcaccgccagcctggtcctggggaagcccctggaagaggtgggcaaggaggaccggcaactggccaaggcgctgaacttcgggcttctctacgggctgggggcggaagggctccggaggtacgccctcaccgcctacggggtgaagctcaccctcgaggaggcccagaagcttcgggacgcgttcttccgggcttaccccgccctgaagcgctggcaccggtcccagcctgagggggaggtggtggtgaggaccctcttgggccggaggaggaccacggaccgctacacggaaaagctcaacaccccggtacagggaaccggggcggacgggctcaagatggccctggccctcctctgggagaaccggggcctactctggggagccttccccgtcctggcggttcacgacgaggtggtgctggaggcccccgaggagggggccaaggagtacctggaaaccctcaccgccctcatgcgccgggggatggaggcggtgcttgggggcgcggtgcccgtggaggtggaaggaggcatctaccgggactggggggccacgccgtgggaggaggcatga

SEQ ID NO: 3 - DNA, 1689 bp, Unknown
  Environmental Sample
gtggaggggtttgaactccactacatcccggaagtaggccccggcatgggggagcttttggacctcctcatgcgccagcccgtcctgggggtggacctggaaaccacggggcttgacccccacaccgcacgccccaggctcctctctctggccggggagcggtttgccgtggtggtggacctcttccgggtgccccttgaagtcttccgccccctcttctcctgggaggaggggccccttttggtggggcacaacctcaagtttgacctcctcttcctcctcaaggccggggtgtggcggggaagcggcagaaggctttgggacaccggactggcccaccaggtgcttcacgcccaagcccgcatgcccgccctcaaggacttagcgccggggctagacaagaccctgcagacctcggactggagcggccccctctccacggaacaggtggcctacgccgcccttgacgcggtggtgcccctctccctctacggggagcagaagaagcgggcccgggccatggggcttgagaaggtccttgaggtggagcaccgcgcccttcccgccgtggcgtggatggagcttaagggggtgcccttcgccccggaactctgggaggaggccgccagggaagcggaacgggaggcggaagccctacgcgcggaactccccttcggggtgaactggaacagccccgcccaggtgctggcctacctgaagggggagggtttggacctccccgacacccgggaggacaccctggccggctaccgggagcaccccctggtggccaagctcctccggtaccgggaagcggccaagcgggtgagcacctacgggaaggagtgggccaagcacctgaacccggccacgggacgcatacacccttcctggcaacagataggggcggaaacgggccgcatggcttgccggaagcccaaccttcagcaggtgccccgggatcccgccctgagaagggccttccggcctaaggaggggcgggtcatgctcaaggccgacttctcccagattgagctacggattgccgccgccatagccaaggaggggcggatgctcagggcgttccgggaggggaaggacctccacgccctcaccgccagcctggtcctggggaagcccctggaagaggtgggcaaggaggaccggcaactggccaaggcgttgaacttcggacttctctacgggctgggggcggaagggctccggaggtacgccctcaccgcctacggggtgaagctcacccccgaggaggcccagaagcttcgggacgcgttcttccgggcttaccccgccctgaagcgctggcaccggtcccagcctgagggggaggtggtggtgaggaccctcttgggccggaggaggaccacggaccgctacacggaaaagctcaacaccccggtacagggaaccggggcggacgggctcaagatggccctggccctcctctgggagaaccggggcctactctggggagccttccccgtcctggccgtgcatgacgaggtggtgcttgaggcccccgaggaaggggccagggagtacctggaagccctcaccgccctcatgcgccaagggatgggagaggtgcttgggggcgcggtgcccgtggaggtggaaggaggcatctaccgggactggggggccacgccgtgggaggaggaggcatga

SEQ ID NO: 4 - protein, 561 aa, Thermus antranikianii
VEGFELHYIPEVGPGMGELLDLLMRQPVLGVDLETTGLDPHTSRPRLLSLAMPGAVVVFDLFGVPLEVFYPLFSREEGPLLVGHNLKFDLLFLLKAGVWRASGKRLWDTGLAHQVLHAQARMPALKDLAPGLDKTLQTSDWGGPLSSEQVAYAALDAAVPLVLYREQRERARTLRLEKVLEVERRALPAVAWMELRGVPFAPELWEEAAREAEREAEALRGELPFGVNWNSPAQVLAYLKGEGLDLPDTREDTLAGYREHPLVAKLLRYREAAKRVSTYGKEWAKHLNPATGRIHPSWQQIGAETGRMACRKPNLQQVPRDPALRRAFRPKEGRVMLKADFSQIELRIAAAIAKEGRMLRAFREGKDLHALTASLVLGKPLEEVGKEDRQLAKALNFGLLYGLGAEGLRRYALTAYGVKLTLEEAQKLRDAFFRAYPALKRWHRSQPEGEVVVRTLLGRRRTTDRYTEKLNTPVQGTGADGLKMALALLWENRGLLWGAFPVLAVHDEVVLEAPEEGAKEYLETLTALMRQGMEEVLGGAVPVEVEGGIYRDWGATPWEEA

SEQ ID NO: 5 - protein, 561 aa, Thermus brockianus
MEGFELHYIPEVGPGMGELLDLLMRQPVLGVDLETTGLDPHTARPRLLSLAGERFAVVVDLFRVPLEVFRPLFSWEEGPLLVGHNLKFDLLFLLKAGVWRGSGRRLWDTGLAHQVLHAQARMPALKDLAPGLDKTLQTSDWGGPLSSEQVAYAGLDAVVPLSLYGEQKKRARAMGLEKVLEVEHRALPAVAWMELRGVPFAPELWEEAAREAEREAKALRAELPFGVNWNSPAQVLAYLKGEGLDLPDTREDTLAGYREHPLVAKLLRYREAAKRVSTYGKEWAKHLNPATGRIHPSWQQIGAETGRMACRKPNLQQVPRDPALRRAFRPPEGKVLLKADFSQIELRIAAAIAREGRMLQAFREGKDLHALTASLVLGKPLEEVGKEDRQLAKALNFGLLYGLGAEGLRRYALTAYGVKLTLEEAQKLRDAFFRAYPALKRWHRSQPEGEVVVRTLLGRRRTTDRYTEKLNTPVQGTGADGLKMALALLWENRGLLWGAFPVLAVHDEVVLEAPEEGAKEYLETLTALMRRGMEAVLGGAVPVEVEGGIYRDWGATPWEEA

SEQ ID NO: 6 - protein, 562 aa, Unknown
  Environmental sample
VEGFELHYIPEVGPGMGELLDLLMRQPVLGVDLETTGLDPHTARPRLLSLAGERFAVVVDLFRVPLEVFRPLFSWEEGPLLVGHNLKFDLLFLLKAGVWRGSGRRLWDTGLAHQVLHAQARMPALKDLAPGLDKTLQTSDWSGPLSTEQVAYAALDAVVPLSLYGEQKKRARAMGLEKVLEVEHRALPAVAWMELKGVPFAPELWEEAAREAEREAEALRAELPFGVNWNSPAQVLAYLKGEGLDLPDTREDTLAGYREHPLVAKLLRYREAAKRVSTYGKEWAKHLNPATGRIHPSWQQIGAETGRMACRKPNLQQVPRDPALRRAFRPKEGRVMLKADFSQIELRIAAAIAKEGRMLRAFREGKDLHALTASLVLGKPLEEVGKEDRQLAKALNFGLLYGLGAEGLRRYALTAYGVKLTPEEAQKLRDAFFRAYPALKRWHRSQPEGEVVVRTLLGRRRTTDRYTEKLNTPVQGTGADGLKMALALLWENRGLLWGAFPVLAVHDEVVLEAPEEGAREYLEALTALMRQGMGEVLGGAVPVEVEGGIYRDWGATPWEEEA

SEQ ID NO: 7 - protein, 832 aa, Thermus aquaticus
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLCCCDPNLQNIPVRTPLGQRIRRGFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 8 - protein, 580 aa, Bacillus stearothermophilus
AAMAFTLADRVTEEMLADKAALVVEVVEENYHDAPIVGIAVVNEHGRFFLRPETALADPQFVAWLGDETKKKSMFDSKRAAVALKWKGIELCGVSFDLLLAAYLLDPAQGVDDVRAAAKMKQYEAVRPDEAVYGKGAKRAVPDEPVLAEHLVRKAAAIWELERPFLDELRRNEQDRLLVELEQPLSSILAEMEFAGVKVDTKRLEQMGKELAEQLGTVEQRIYELAGQEFNINSPKQLGVILFEKLQLPVLKKTKTGYSTSADVLEKLAPYHEIVENILHYRQLGKLQSTYIEGLLKVVRPDTKKVHTIFNQALTQTGRLSSTEPNLQNIPIRLEEGRKIRQAFVPSESDWLIFAADYSQIELRVLAHIAEDDNLMEAFRRDLDIHTKTAMDIFQVSEDEVTPNMRRQAKAVNFGIVYGISDYGLAQNLNISRKEAAEFIERYFESFPGVKRYMENIVQEAKQKGYVTTLLHRRRYLPDITSRNFNVRSFAERMAMNTPIQGSAADIIKKAMIDLNARLKEERLQAHLLLQVHDELILEAPKEEMERLCRLVPEVMEQAVTLRVPLKVDYHYGSTWYDAK

SEQ ID NO: 9 - protein, 928 aa, Escherichia coli [sequence omitted]

SEQ ID NO: 10 - protein, 574 aa, Aquifex aeolicus
MDFEYVTGEEGLKKAIKRLENSPYLYLDTETTGDRIRLVQIGDEENTYVIDLYEIQDIEPLRKLINERGIVGHNLKFDLKYLYRYGIFPSATFDTMIASYLLGYERHSLNHIVSNLLGYSMDKSYQTSDWGASVLSDAQLKYAANDVIVLRELFPKMRDMLNELDAERGEELLKTRTAKIFDLKSPVAIVEMAFVREVAKLEINGFPVDVEELTNKLKAVERETQKRIQEFYIKYRVDPLSPKQLASLLTKKFKLNLPKTPKGNVSTDDKALTSYQDVEPVKLVLEIRKLKKIADKLKELKEHLKNGRVYPEFKQIGAVTGRMSSAHPNIQNIHRDMRGIFKAEEGNTFVISDFSQIELRIAAEYVKDPLMLDAFKKGKDMHRYTASVVLGKKEEEITKEERQLAKAINFGLIYGISAKGLAEYAKLGYGVEISLEEAQVLRERFFKNFKAFKEWHDRVKKELKEKGEVKGHTLLGRRFSANTFNDAVNYPIQGTGADLLKLAVLLFDANLQKKGIDAKLVNLVHDEIVVECEKEKAEEVKEILEKSMKTAGKIILKEVPVEVESVINERWTKD

SEQ ID NO: 11 - protein, 5 aa, Artificial Sequence
  Synthetic peptide
XXXXX

SEQ ID NO: 12 - protein, 5 aa, Artificial Sequence
  Synthetic peptide
EXXRR

SEQ ID NO: 13 - protein, 24 aa, Artificial Sequence
  Synthetic peptide
XFGXXYGXXXXXXXXXXXXXXXXX

SEQ ID NO: 14 - protein, 24 aa, Artificial Sequence
  Synthetic peptide
XFGXXYGXXXEXXRRXXXXXXXXK

SEQ ID NO: 15 - protein, 26 aa, Artificial Sequence
  Synthetic peptide
XFGXXYGXXXXXXXXYXXXXYGXXXX

SEQ ID NO: 16 - protein, 26 aa, Artificial Sequence
  Synthetic peptide
XFGXXYGXXXXGXXXYAXXXYGVXXX

SEQ ID NO: 17 - protein, 26 aa, Artificial Sequence
  Synthetic peptide
NFGLLYGLGAEGLRRYALTAYGVKXX

SEQ ID NO: 18 - protein, 15 aa, Artificial Sequence
  Synthetic peptide
LKADFSQIELRIAAA

SEQ ID NO: 19 - DNA, 26 bp, Artificial Sequence
  Synthetic primer
gccgccgactactcccarathgarht

SEQ ID NO: 20 - DNA, 27 bp, Artificial Sequence
  Synthetic primer
cangtrctrctctaccacaagctcccg

SEQ ID NO: 21 - DNA, 35 bp, Artificial Sequence
  Synthetic primer
ggccacgcgtcgactagtacnnnnnnnnnngatat

SEQ ID NO: 22 - DNA, 35 bp, Artificial Sequence
  Synthetic primer
ggccacgcgtcgactagtacnnnnnnnnnnacgcc

SEQ ID NO: 23 - DNA, 20 bp, Artificial Sequence
  Synthetic primer
ggccacgcgtcgactagtac

SEQ ID NO: 24 - DNA, 24 bp, Artificial Sequence
  Synthetic primer
acgccctcaccgccagcctggtcc

SEQ ID NO: 25 - DNA, 26 bp, Artificial Sequence
  Synthetic primer
ttctcccagaggagggccagggccat

SEQ ID NO: 26 - DNA, 34 bp, Artificial Sequence
  Synthetic primer
cgaattccatatggaggggtttgaactccactac

SEQ ID NO: 27 - DNA, 29 bp, Artificial Sequence
  Synthetic primer
cgcagatcttcatgcctcctcccacggcg

SEQ ID NO: 28 - protein, 14 aa, Artificial Sequence
  Synthetic peptide
XXXXXXXXXXXXXX

SEQ ID NO: 29 - protein, 16 aa, Artificial Sequence
  Synthetic peptide
XXXXXYXXXXYXXXXX

SEQ ID NO: 30 - protein, 16 aa, Artificial Sequence
  Synthetic peptide
XGXXXYAXXXYGXXXX

(19) United States
(12) Patent Application Publication
(10) Pub. No.: US 2008/0311626 A1
(43) Pub. Date: Dec. 18, 2008

(54) Dna Polymerases Having Strand Displacement Activity

(75) Inventors: Sigridur Hjorleifsdottir, Reykjavik (IS); Sveinn Ernstson, Reykjavik (IS); Thorarinn Blondal, Gardabaer (IS); Arnthor Aevarsson, Hveragerdi (IS); Gudmundur Oli Hreggvidsson, Reykjavik (IS); Jakob Kristjansson, Gardabaer (IS)

Correspondence Address: BIRCH STEWART KOLASCH & BIRCH, PO BOX 747, FALLS CHURCH, VA, 22040-0747, US

(73) Assignee: Arkea hf, Reykjavik (IS)

(21) Appl. No.: 11/662,879

(22) PCT Filed: Sep. 19, 2005
(86) PCT No.: PCT/IS05/00022

§ 371 Date: Jul. 28, 2008

(30) Foreign Application Priority Data
Sep. 17, 2004 (IS) 7461

(51) Int. Cl. C12P 19/34; C12N 9/10; C07H 21/04; C12N 1/21

(52) U.S. Cl. 435/91.2; 435/193; 536/23.2; 435/252.3

(57) ABSTRACT

The invention provides novel strand displacement DNA polymerases which can be used in a rapid and efficient strand displacement amplification reactions. The polymerases are significantly more thermostable than prior art polymerase and retain high activity at elevated temperatures. Also disclosed are genes encoding the polymerases and vectors comprising the genes. Representative polymerases of the invention are obtainable from bacterial strains of the species Thermus antranikianii and Thermus brockianus and also from environmental samples with isolation of the source species.

### BACKGROUND OF THE INVENTION

DNA polymerase is an enzyme capable of catalyzing replication of DNA. Being fundamental to basic biology, DNA polymerases have received great attention and numerous functional and structural studies have given a detailed insight into the complex structure-function relationship in this group of enzymes. Due to their abilities to replicate genetic material, DNA polymerases have also become indispensable research tools.

A polypeptide having DNA polymerase activity may have other activities including 3′-5′ exonuclease activity (proofreading activity) and 5′-3′ exonuclease activity. Active sites, conferring the separate fundamental activities, have been mapped to different domains illustrated for example by the structure of Thermus aquaticus DNA polymerase having an N-terminal 5′-3′ exonuclease domain, a 3′-5′ exonuclease middle domain followed by the polymerase domain. The polymerase domain is commonly further characterized by smaller structural features, such as so called palm, fingers and thumb, each shown to have specific roles in the function of the enzyme. The fundamental activities of DNA polymerases are affected by the properties of the enzyme which are greatly variable depending on the source of the enzyme. Characterized DNA polymerases thus display a wide spectrum in terms of their abilities and properties such as optimal working temperature, thermostability, processivity and fidelity (for a review see Brautigam and Steitz 1998b; Steitz 1999).

The use of thermostable enzymes, foremost thermostable DNA polymerases, has revolutionized the field of recombinant DNA technology and such enzymes are of great importance in the research industry today. DNA polymerases are being used for a variety of biological applications including sequencing and amplification of nucleic acids such as by the polymerase chain reaction (PCR) requiring thermal cycling or through isothermal amplification. A large number of DNA polymerases have been identified and described and shown to have varying suitability for different applications. Many DNA polymerases have also been modified in different ways such as through truncations and site-directed mutagenesis to alter their properties including alterations to abolish basic activities such as the 3′-5′ exonuclease activity. DNA polymerases have been described from a number of Thermus species including DNA polymerase I from Thermus aquaticus (Taq DNA polymerase) which is widely used in PCR amplification due to the thermostability of the enzyme (Saiki et al. 1988).

In vitro amplification of genetic material, including whole genome amplification, is becoming increasingly important. Genotyping techniques are for example used for determination and screening of single nucleotide polymorphism (SNP) requiring a certain amount of genomic DNA from different individuals. However, the amount of available DNA, such as from clinical samples, is often limited. Whole genome amplification is thus becoming essential for generating sufficient amount of DNA for analysis and to renew genetic material from an original sample.

Many methods have been developed for amplification of genetic material in vitro, such as whole genome amplification. Several of the methods are based on PCR technology and successfully used for many purposes. Certain shortcomings of the PCR-based methods have been overcome by the development of methods based on the use of strand displacement DNA polymerases. This includes methods termed for example “Rolling Circle Amplification” using circular DNA templates such as cloning vectors (Nelson et al. 2002; Dean et al. 2001; Alsmadi et al. 2003, Detter et al. 2002), “Hyperbranched Strand, Displacement Amplification” (Lage et al. 2003) and “Multiple Displacement Amplification” (Dean et al. 2002; U.S. Pat. No. 6,617,137 B2; U.S. Pat. No. 6,124,120). This technology has provided powerful methods for relatively simple whole genome amplification generating long DNA products with close to unbiased representation of the genome being amplified. These methods have considerable advantages over other methods based on thermocycling protocols. For example, the strand displacement methods generally produce larger fragments with higher yield and less sequence bias than PCR-based methods. In contrast, the PCR-based methods utilizing thermocycling protocols typically give product of short length with incomplete coverage and biased representation of the genome by favoring amplification of certain regions in the genome (Lasken and Egholm 2003; Paez et al. 2004; Lage et al. 2003).

Multiple Displacement Amplification offers several advantages. Large amounts of material can be produced even from very small amounts of starting material. The material produced consists of relatively long DNA products, averaging 12 kb, with unbiased coverage of the starting material as mentioned above. Also, MDA can be carried out using crude samples, such as clinical samples consisting of cell or blood lysates. Yields of DNA can be independent of the amount of starting material and thus avoids the need for determination the concentration of DNA and adjustment of the concentration prior to subsequent analysis. MDA offers the possibility of alternative and simplified sampling of genetic material as less material is needed to start with and this can for example simplify the collection of samples from human patients in clinical settings. Furthermore, MDA lends itself relatively easily to automation (Lasken and Egholm 2003).

The success of MDA and methods based on strand displacement during DNA synthesis is dependent on the properties of the DNA polymerase being used for the amplification. DNA polymerases are widely different with respect to the ability to displace existing DNA strand as a new strand is being synthesized. The most suitable DNA polymerase that has been found and tested to date for this purpose is Bacteriophage Phi29 DNA polymerase according to the present state of the art is generally the enzyme of choice for these methods based on unusual and advantageous properties of this polymerase (see Technical Reference sheet, New England Biolabs Inc.). Phi29 DNA polymerase has very tight binding to the DNA substrate giving very high processivity and ability to generate very long DNA products up to more than 100 kb. The essential feature of the enzyme is the ability to synthesize a new DNA strand and at the same time displace previously made DNA strands from the template strand. This is thought to proceed through a mechanism producing hyperbranched product from the starting material as DNA strands are being displaced and becoming new starting points for synthesis of new strands. Phi29 DNA polymerase originates from a mesophilic bacteriophage and the enzyme is normally used at about 30° C. in an isothermal reaction. Therefore, avoiding thermocycling and the ability of phi29 DNA polymerase to synthesize through difficult regions in the template material results in even representation of the starting genetic material (Dean et al. 2001, Blanco and Salas 1996, Blanco et al. 1989).

Amplification of genetic material becomes critical in situations of limited supply of the material. Strand displacement amplification has become an important technique for amplification of genetic material of limited quantity. DNA polymerases with strand displacement activity are known in the art such as disclosed in U.S. Pat. No. 5,744,312. Seemingly, Phi29 DNA polymerase, and, to a lesser extent, the large fragment of Bacillus stearothermophilus DNA polymerase are to date the most commonly used and most suitable DNA polymerases for strand displacement amplification not requiring thermal cycling (Technical reference, New England Biolabs Inc). The underlying ability for amplification without thermal cycling is based on strand-displacement properties of these polymerases where assumingly the DNA polymerase is able to displace annealed non-template strand and synthesize a new strand whereas conventional DNA polymerases such as Thermus aquaticus DNA polymerase would normally be hindered by the presence of a non-template strand annealed to the template strand. However, Phi29 DNA polymerase is apparently not a very efficient enzyme compared to conventional DNA polymerase such as Taq DNA polymerase in terms of speed and thus the yield of material produced after a certain time.

### SUMMARY OF THE INVENTION

Provided by the present invention are novel thermostable DNA polymerases which preferably have DNA strand displacement activity and can be used in a rapid and efficient strand displacement amplification reactions. Compared to Phi29 DNA polymerase, the DNA polymerases provided by the invention are much more efficient and have other distinctive advantageous properties such as the ability to work at higher temperatures. Enzymes of the type provided by the invention may proof to be valuable tools in various applications in recombinant DNA technology and other molecular biology procedures.

The present invention relates to isolated polypeptides having strand-displacement DNA polymerase activity and active derivatives or fragments thereof (i.e. fragments and derivatives retaining the DNA polymerase activity of the parent polypeptide from which they are derived) as well to their use in amplification of genetic material, including amplification for genetic analysis, for example genotyping. The invention encompasses the polypeptides having the amino acid sequences shown as SEQ ID NO: 4, SEQ ID NO: 5 and SEQ ID NO: 6 and polypeptides having strand displacement DNA polymerase activity with substantially similar amino acid sequences to said sequences as well as active derivatives or fragments thereof. The invention further pertains to nucleic acids encoding the polypeptides of the invention, including the nucleic acid sequences depicted as SEQ ID NO: 1, SEQ ID NO: 2 and SEQ ID NO: 3. The invention also pertains to DNA constructs containing the isolated nucleic acid molecules described herein operatively linked to a regulatory sequence; and to host cells comprising the DNA constructs.

The invention provides in one aspect an isolated thermostable polypeptide belonging to the DNA polymerase A family as further defined herein and in more detail in herein referenced articles, which polypeptide is encoded by a gene sequence obtainable from a Thermus sp. and has a non-truncated molecular weight in the range of about 58-68 kDa (kiloDaltons), such as in the range of about 61-65 kDa, including about 61, 62, 63, 64, or 65 kDa. The polypeptides preferably have DNA polymerase strand-displacement activity.

In certain embodiments, the invention relates to isolated thermostable polypeptides having strand displacement DNA polymerase activity, which are obtainable from strains identified as Thermus antranikianii (strain 2120) and Thermus brockianus (strain 140). Also provided is an isolated polypeptide encoded by a gene isolated from a complex environmental biomass sample. Isolated polypeptides provided by the invention can replace DNA polymerases, such as Phi29 DNA polymerase, in applications that utilize strand displacement activities of a DNA polymerase, in particular in applications that require and/or benefit from elevated temperatures (above about 50° C.). The polypeptides of the present invention may also be used in other applications, in particular applications that require elevated temperatures (above about 50° C.).

In one embodiment of the invention, isolated thermostable polypeptides having strand displacement DNA polymerase activity provided by the invention refer to a novel DNA polymerase from the thermophilic bacteria of the species Thermus antranikianii and Thermus brockianus. Compared to known DNA polymerase from the genus Thermus, the polypeptides provided by the invention have analogous activity but novel properties and structure. The polypeptides having strand displacement DNA polymerase activity provided by the invention comprise a polymerase domain and a 3′-5′ exonuclease domain but naturally lack a 5′-3′ exonuclease domain.

The polypeptides of the invention have been found to be significantly more thermostable than some other polypeptides known in the prior art and those which are currently most commonly used for isothermal amplification of genetic material, in particular DNA polymerase from bacteriophage Phi29. The enhanced stability of the polypeptides provided by the invention allow their use under temperature conditions which would be prohibitive for other analogous enzymes such as bacteriophage Phi29 DNA polymerase, thereby increasing the range of conditions which can be employed and also the type of methods that can be used. Additionally, the polypeptides of the invention have other different functional properties that can be advantageous in certain applications, compared to other homologous polypeptides known from the prior art.

The invention further pertains to the use of the polypeptides provided by the invention in various applications including strand displacement amplifications such as rolling circle amplification (Nelson et al. 2002; Dean et al. 2001; Alsmadi et al. 2003, Detter et al. 2002) and multiple displacement amplification (Nelson et al. 2002; Dean et al. 2001; Alsmadi et al. 2003, Detter et al. 2002).

The invention pertains to methods using DNA polymerases of the invention for DNA synthesis by addition of deoxynucleotides to the 3′ end of a polynucleotide chain, using a complementary nucleic acid strand as a template and displacement intervening strands of nucleic acids hybridized to the template strand. The invention thus pertains to amplification of genetic material such as amplification of genomic DNA.

Also provided by the invention are kits for practicing the subject methods. In further describing the subject invention, the subject methods will be discussed first in greater detail followed by a description of the kits for practicing the subject methods.

A thermostable polypeptide having DNA polymerase strand displacement activity of the present invention is suitably selected from the group consisting of: a thermostable polypeptides DNA polymerase strand displacement activity obtained from a Thermus species; a polypeptide comprising the amino acid sequence of SEQ ID NO: 4, SEQ ID NO: 5 or SEQ ID NO: 6; a polypeptide encoded by a nucleic acid comprising the sequence of SEQ ID NO: 1, SEQ ID NO: 2 and SEQ ID NO: 3; a polypeptide having at least 40% sequence identity with the amino acid sequence of SEQ ID NO: 4, SEQ ID NO: 5 or SEQ ID NO: 6; or an active fragment or derivative thereof.

The thermostable polypeptides having DNA polymerase strand displacement activity described herein have advantageous properties in comparison to prior art strand displacement DNA polymerases, such as very efficient strand displacement activity combined with thermostability and proof-reading activity. In a preferred embodiment, the methods of the invention are performed at temperatures in the range of about 50° C. up to about 95° C.

### BRIEF DESCRIPTION OF THE DRAWINGS

The foregoing and other objects, features and advantages of the invention will be apparent from the following more particular description of preferred embodiments of the invention, as illustrated in the accompanying drawings.

FIG. 1: shows a phylogenic tree of the amino acid sequences of DNA polymerase Pol-11 (SEQ ID NO: 4), DNA polymerase Pol-3 (SEQ ID NO: 5), and DNA polymerase Pol-62 (SEQ ID NO: 6), together with selected prior art DNA polymerase I sequences. All the sequences are of DNA polymerases of family A except phi29 DNA polymerase which belongs to family B and is used here as an outgroup. The accession numbers of the public sequences are as follows: Thermus flavus P30313; Thermus filiformis 052225; Thermus thermophilus P52028; Thermus aquaticus P19821; Geobacillus stearothermophilus AAB62092; Thermotoga maritima NP229419; Thermomicrobium roseum AAO85272; Desulfitobacterium hafniense ZP_{—}00097788; Aquifex pyrophilus AAO15360; Aquifex aeolicus NP_{—}214348; Bacteriophage phi29 X53370.

The DNA polymerases of the invention are distantly related to all prior art DNA polymerases and clearly form a distinct branch on the phylogenetic tree.

FIG. 2: shows activity of DNA polymerase Pol-11 and DyNAzyme DNA polymerase measured as incorporation of labelled nucleotides at 55° C. Total CPM was 15585.

FIG. 3: shows activity of Pol-11 and DyNAzyme (DZ) as a function of temperature.

FIG. 4: shows activity of Pol-11 and DyNAzyme (DZ) as % incorporation of labelled nucleotides (3H-dTTP) over a time period of 120 minutes, measured at 55° C.

FIG. 5: shows activity of Pol-11 and Pol-3 at 55° C. for different time periods. DyNAzyme (DZ) is a control sample. Total CPM was 11032.

FIG. 6: shows activity as function of pH for Pol-11 and DyNAzyme (DZ).

FIG. 7: shows the effects of varying the MgCl₂ concentration (shown in mM on x-axis) in activity measurements of Pol-11 at 55° C. for 10 min. DyNAzyme (DZ) is used for comparison.

FIG. 8: shows the effects of varying the (NH₄)₂SO₄ concentration (shown in mM on x-axis) in activity measurements of Pol-11 at 55° C. for 10 min. DyNAzyme (DZ) is used for comparison.

FIG. 9: shows the relative activity of Pol-11 DNA polymerase at different temperatures with and without 0.5 M L-Proline.

FIG. 10: shows heat inactivation (thermostability) of Pol-11 with and without L-Proline as stabilization agent. After 15 min incubation at 94° C. the Proline reaction mixture had between 2-3 fold activity (assayed at 55° C.) compared to the untreated mixture.

FIG. 11: shows the effects of doubling the amount of template DNA and labelled nucleotide (dNTP) in the reaction mixture for Pol-11 and DyNAzyme (DZ). Total CPM was 15.798 for 1×3H and 33.625 for 2×3H.

FIG. 12: shows the activity of Pol-11 starting with standard amount of template DNA (1×DNA), two times the standard amount (2×DNA) and by adding more template DNA at 30 and 60 min.

FIG. 13: illustrates purification of Pol-11 on HiTrap Chelatin HP column. Lanes 8, 9 and 10 show final fractions. Lanes contain 1: ladder, 2: pol-3 (10 min 65° C.), 3: pol-11 (10 min 65° C.), 4: A10, 5: A12, 6: B2, 7: B5, 8: C1, 9: C6, 10: D1.

FIG. 14: Amplification of plasmid DNA

A) Amplification of pUC19 plasmid DNA with Pol-11 or Phi29. Plasmid DNA was amplified for 14 hours with Pol-11 at 55° C. in the presence of specific primers (lanes 2-4) or absence of primers (lane 1). Same amount of template was amplified with for 14 hours with phi 29 at 30° C. in the presence of specific primers (lane 5). Lane 6 contains a size marker (1 kb ladder from NEB).
B) Same samples as in A but heated at 96° C. for 10 minutes prior to load on agarose gel. Lane 6 contains a size marker (1 kb ladder from NEB).

FIG. 15: Nucleotide requirements of pol 11 amplification. 10 ng of pUC 19 plasmid DNA was treated with pol 11 at 55° C. for 14 hours with different primer and nucleotide compositions. Lanes 2-5 contain dATP and dTTP, lanes 6-9 dGTP and dCTP, lanes 10-13 dNTP, and lanes 14-17 none. Reactions in lanes 2, 6, 10 and 14 received no primers, other reactions received specific primers. The plasmid band in all lanes is probably supercoiled DNA and does not participate in any reaction. Amplification occurs in absence of primers (lane 10) starting from nicked relaxed plasmid DNA.

FIG. 16:

Exonuclease activity of Pol-11 DNA polymerase
Column 1: Pol-11 0.25 ss DNA; dNTP
Column 2: Pol-11 1.0 ss DNA; dNTP
Column 3: Pol-11 3.0 ss DNA; dNTP
Column 4: ss DNA; dNTP
Column 5: Pol-11 0.25 ds DNA
Column 6: Pol-11 1.0 ds DNA
Column 7: Pol-11 3.0 ds DNA
Column 8: ds DNA
Column 9: Pol-11 0.25 ds DNA; dNTP
Column 10: Pol-11 1.0 ds DNA; dNTP
Column 11: Pol-11 3.0 ds DNA; dNTP
Column 12: ds DNA; dNTP
Column 13: Pol-11 0.25 ds DNA
Column 14: Pol-11 1.0 ds DNA
Column 15: Pol-11 3.0 ds DNA
Column 16: ds DNA
Column 17: ss DNA—untreated
Column 18: ds DNA—untreated

FIG. 17: shows a gel demonstrating amplification of human DNA using thiolhexamers and Pol-11. Reactions in lanes 1 3 5 7 received 1 ng human DNA as starting material for Pol-11 amplification. Lanes 2 4 6 8 received 5 ng human DNA as starting material. The reactions were subjected to 5 minute amplifications cycles at 55° C. interrupted by 30 second annealing steps at 30° C. Lanes 1-2 were subjected to 5 amplification cycles), lanes 3-4 to 10 cycles and lanes 5-8 to 20 cycles respectively. Reactions in lanes 7 and 8 received no enzyme. Prior to addition of enzyme the samples were heated at 94° C. for 4 minutes.

FIG. 18: Amplification from hexamer amplified human genomic DNA. PCR results from human DNA template amplified with pol 11. Marker gene: Beta-actin. 1 ul of 20 ul reactions in FIG. 17 used as template in lanes 1-8. Lanes 1-8 same reactions as in FIG. 17. Lanes 9-12 and 14-17 are PCR reactions that received untreated human DNA as template, 5; 2.5; 1.25; 0.6; 1; 0.5; 0.25 and 0.125 ng respectively. Lane 13 contains a size marker (1 kb ladder from New England Biolabs).

FIG. 19: shows PCR products from amplified human genomic DNA.

FIG. 20: The activity for Pol-11 on activated DNA using 0.06 micrograms protein over time with 0.1 mg/ml DNA (diamonds) or 0.6 mg/ml DNA (squares).

FIG. 21: Activity of 0.02 microgram and 0.1 micrograms of Pol-11 and Phi29 DNA polymerases respectively. Specific activity after 10 minutes corresponds to about 360.000 units per mg for Pol-11 and 10.800 units per mg for Phi 29 DNA polymerase. Y-axis shows percent of total incorporation.

FIG. 22: shows the amino acid sequence alignment of selected DNA polymerase sequences. Taq is DNA polymerase I from Thermus aquaticus (accession number 1TAQ), Bst is DNA polymerase from Bacillus stearothermophilus (accession number 2BDP_A), Eco is DNA polymerase I from Echerichia coli (accession number P00582), Aea is DNA polymerase I from Aquifex aeolicus (accession number 067779), Pol-11 is SEQ ID NO: 4; Pol-3 is SEQ ID NO: 5 and Pol-62 is SEQ ID NO: 6. The top three sequences have been truncated at the N-terminal and thus not showing the 5′-3′ exonuclease domain which is naturally absent in the other sequences including the sequences of the invention. Locations of sequence motifs in the 3′-5 exonuclease domain are indicated (Exo I, Exo ii and Exo III) as well as sequence motifs in the polymerase domain (Motif A, Motif B and Motif C). The sequence alignment was created using automatic alignment with program ClustalX (ref) followed by some manual adjustments, mainly in the exonuclease domain, using additional information of described sequence motifs and structure information. The sequences of Pol-11, Pol-3 and Pol-62 are most similar to the Aquifex sequence although the similarity is limited. The Aquifex sequence has similar sequence identity to all three sequences of the invention, for example 33% with respect to the Pol-11 sequence, calculated as percentage of identical matches between the two sequences over the aligned region including any gaps in the length.

### DETAILED DESCRIPTION OF THE INVENTION

As used herein, the term “nucleic acid” encompasses the terms “oligonucleotide” and “polynucleotide” and means single-stranded or double-stranded polymers of nucleotide monomers, including 2′-deoxyribonucleotides (DNA) and ribonucleotides (RNA). The nucleic acid can be composed entirely of deoxyribonucleotides, entirely of ribonucleotides, or chimeric mixtures thereof, linked by internucleotide phosphodiester bond linkages, and associated counter-ions, e.g., H⁺, NH₄⁺, trialkylammonium, Mg²⁺, Na⁺ and the like. The nucleic acid may also be a peptide nucleic acid (PNA) formed by conjugating bases to an amino acid backbone. The term also refers to nucleic acids containing modified bases.

The term “primer” normally refers herein to an oligonucleotide used, for example in amplification of nucleic acids such as PCR. The primer can be comprised of unmodified and/or modified nucleotides, for example modified by a biotin group attached to the nucleotide at the 5′ end of the primer. The primer may contain at least 15 nucleotides, and preferably at least 18, 20, 22, 24 or 26 nucleotides.

The term “fragment” is intended to encompass a portion of a nucleic acid or a protein. A nucleic acid fragment may be at least about 15 contiguous nucleotides, preferably at least about 18, 20, 23 or 25 nucleotides, and can be 30, 40, 50, 100, 200 or more nucleotides in length. A protein fragment may be at least about 5 contiguous amino acids in length, preferably at least about 7, 10, 15, or 20 amino acids, and can be 25, 30, 40, 50 or more amino acids in length. A particularly useful protein fragment is one that retains activity, for example enzyme activity, cofactor binding capability, ability to bind other proteins, such as receptors, or ability to bind DNA.

The term, “polypeptide”, as used herein, refers to polymers of amino acids linked by peptide bonds and includes proteins, enzymes, peptides, and other gene products encoded by nucleic acids described herein.

The term “isolated” as used herein means that the material is removed from its original environment (e.g. the natural environment where the material is naturally occurring). For example, a polynucleotide or polypeptide while present in a living source organism is not isolated, but the same polynucleotide or polypeptide, which is separated from some or all of the coexisting materials in the natural system, is isolated. Such polynucleotides could for example be part of a vector and/or such polynucleotides or polypeptides could be part of a composition, and still be isolated in that the vector or composition is not part of the natural environment. When referring to a particular polypeptide, the term “isolated” refers to a preparation of the polypeptide outside its natural source and preferably substantially free of contaminants.

“Thermostable” is defined herein as having the ability to withstand high temperatures such as above about 60° C. for at least 30 minutes while retaining substantial enzymatic activity, at preferred temperatures above 50° C., such as between 50° C. and 100° C., at preferred temperatures of about 50° C. to about 75° C. and at even more preferred temperatures of about 55° C. to about 70° C.

“Thermophilic bacteria”, also referred to as “thermophiles”, are defined as bacteria having optimum growth temperature above 50° C. “Thermophilic bacteriophages” or “thermostable bacteriophages” are defined as bacteriophages having thermophilic bacteria as hosts.

“Thermophilic isolate” as used herein refers to a bacterial isolate which has been isolated from a high temperature environment and grown and maintained in a laboratory as a pure culture.

Methods of producing replicate copies of the same polynucleotide, such as PCR or gene cloning, are collectively referred to herein as “amplification” or “replication”. For example, single- or double-stranded DNA can be replicated to form another DNA with the same sequence. RNA can be replicated, for example, by RNA directed RNA polymerase, or by reverse transcribing the RNA and then performing a PCR. In the latter case, the amplified copy of the RNA is a DNA with the correlating or homologous sequence.

The polymerase chain reaction (“PCR”) is a reaction in which replicate copies are made of a target polynucleotide using one or more primers, and a catalyst of polymerization, such as a DNA polymerase, and particularly a thermally stable polymerase enzyme. Generally, PCR involves repeatedly performing a “cycle” of three steps: 1) “melting”, in which the temperature is adjusted such that the DNA dissociates to single strands, 2) “annealing”, in which the temperature is adjusted such that oligonucleotide primers are permitted to anneal to their complementary nucleotide sequence to form a duplex at one end of the polynucleotide segment to be amplified; and 3) “extension” or “synthesis”, which can occur at the same or slightly higher and more optimum temperature than annealing, and during which oligonucleotides that have formed a duplex are elongated with a thermostable DNA polymerase. The cycle is then repeated until the desired amount of amplified polynucleotide is obtained. Methods for PCR amplification can be found in U.S. Pat. Nos. 4,683,195 and 4,683,202.

The methods disclosed herein involving the molecular manipulation of nucleic acids are known to those skilled in the art. See generally Ausubel, F. M. et al., “Short Protocols in Molecular Biology,” John Wiley and Sons (1995); and Sambrook, I., et al., “Molecular Cloning, A Laboratory Manual,” 2nd ed., Cold Spring Harbor Laboratory Press (1989).

Bacterial cells normally carry several DNA polymerases, including DNA polymerases I, II and III. DNA polymerase I from several Thermus species, foremost Thermus aquaticus (Taq) DNA polymerase I, has been of paramount importance for recombinant DNA technology including the polymerase chain reaction (PCR). We have discovered an apparently unknown type of DNA polymerase from a number of Thermus strains. Compared to known sequences, this DNA polymerase is most similar to Aquifex sp. DNA polymerase I and consists of a catalytic domain and a 3′-5′ proofreading exonuclease domain but is lacking a 5′-3′ exonuclease domain. Interestingly, the DNA polymerases of the invention have intriguing strand displacement properties and are highly active in isothermal amplification of genetic material with efficiency exceeding other DNA polymerases currently used for isothermal DNA amplification such as the bacteriophage Phi29 DNA polymerase (Dean et al. 2001, Blanco and Salas 1996, Blanco et al. 1989).

The DNA polymerases of the invention were surprisingly discovered from Thermus species which previously have been extensively studied, including as a source of DNA polymerases. Since the first description of the genus Thermus (Brock and Freeze, 1969) seven other species have been validly described (Oshima and Imahori, 1974; Hudson et al., 1987), (Kristjansson et al., 1994; Williams et al., 1995; Williams et al., 1996; Chung et al., 2000). The most widely used DNA polymerase to date, Taq DNA polymerase I, is from Thermus aquaticus and similar DNA polymerases from other Thermus species have been characterized and are available commercially. Other types of DNA polymerases, including DNA polymerase III and DNA polymerase of family X have been identified in Thermus species. However, a DNA polymerase of the type provided by the invention has not been identified in Thermus strains to our knowledge. Furthermore, the whole genome of Thermus thermophilus has been sequenced (Henne et al. 2004) without revealing a gene coding for a DNA polymerase of the same type as provided here. In light of the extensive efforts in the prior art, including a whole genome sequencing of Thermus thermophilus and characterization of its gene products; it was therefore very surprising that a novel type of DNA polymerases was discovered and obtained from Thermus species. Not only are the sequences of the DNA polymerases provided here (SEQ ID NO: 4, 5 and 6) distantly related to any known DNA polymerases but the properties of the DNA polymerases provided here are also unique.

The results disclosed herein surprisingly reveal a new family of DNA polymerases originating from Thermus strains. Our results reveal not only one sequence of the DNA polymerases of the present invention but three non-identical but closely related sequences that can be used to define this new protein family through analysis of structural features and phylogenetic relationships to other known DNA polymerases. Certain structural features of the DNA polymerases of the invention set them apart from other known DNA polymerase originating from Thermus strains as well as from all other known DNA polymerases. The DNA polymerases of the present invention belong to family A DNA polymerases as can be seen through sequence comparisons to public database sequences such as using BLAST algorithm. The prior art contains some family A DNA polymerases originating from Thermus species such as the well known DNA polymerase I from Thermus aquaticus (Taq DNA polymerase) having a full-length molecular weight close to 94 kDa and including a 5′-3′ exonuclease domain. The DNA polymerases of the present invention are clearly distinct from prior art family A type DNA polymerases from Thermus species by the considerable smaller size mainly due to the absence of a 5′-3′ exonuclease domain.

The properties of the enzymes as strand displacing polymerases in amplification of DNA are unlike properties of characterized DNA polymerases previously obtained from Thermus species such as Taq DNA polymerase I and also significantly different from other polymerases from prior art with characterized strand displacement activity, including Bst DNA polymerase fragment and Phi29 DNA polymerase (Technical Sheet, New England Biolabs Inc). The increased efficiency in isothermal amplification of genetic material using the DNA polymerases of the invention, compared to the use of conventional DNA polymerases used for this purpose, provides a significant advantage which can be utilized in numerous state of the art applications and also opens the possibilities for development of new applications.

Taq DNA polymerase was first described 1976 (Chien et al., 1976) and was the first thermophilic enzyme used in the PCR reaction. Taq does not have the 3′-5′ exonuclease activity responsible for the proofreading mechanism like E. coli Pol I, which is of the same family of enzymes (often referred to as DNA polymerase family A). T. thermophilus DNA polymerase has been widely studied (Perler et al., 1996), and in addition to DNA polymerase activity it possesses very efficient reverse transcriptase activity in the presence of MnCl₂ and has for this reason been a very valuable tool in molecular biology research. T. filiformis DNA polymerase was cloned and characterized by Choi et al. (Choi et al., 1999). The commercially available DNA polymerase known as DyNAzyme™ was isolated in our laboratory and described by Mattila and coworkers and believed to be from T. brockianus (Mattila et al., 1993). However, resent results obtained in our laboratory indicate that this Thermus strain was not correctly identified and that the DyNAzyme polymerase is actually from a potentially new Thermus sp. isolated in our laboratory (Skirnisdottir, 2001; Hjorleifsdottir, 2002). Phylogenetic analysis of bacterial strains based on partial sequencing of the SSU rRNA gene has been done successfully in our laboratory (Skirnisdottir et al., 2000; Hjorleifsdottir et al., 2001) and this method has been used to identify the Thermus species.

In some of our earlier experiments, many Thermus strains were screened for the presence of DNA polymerase activity. The results indicated apparent uneven distribution of a particular DNA polymerase activity. In more recent experiments, DNA polymerase genes were directly amplified from a similar spectrum of Thermus strains through the use of degenerate PCR techniques (GENEMINING, Prokaria Ltd., Reykjavik, Iceland). From sequence analysis of the amplified genes emerged evidence for the presence of hitherto unknown type of DNA polymerase. Judging from the success of gene amplification, this new type of DNA polymerase was only found in certain Thermus strains. Interestingly, the gene for this particular type of polymerase was not obtained in either T. aquaticus or T. thermophilus strains but only in certain other strains including strains for the species T. scotoductus, T. brockianus, T. oshimai and T. atranikiani. In addition, the recently published genomic sequence of Thermus thermophilus HB27 (Henne et al. 2004) does not contain a gene for a similar DNA polymerase. This fact, together with the unexpectedly high similarity of the obtained DNA polymerase sequences, suggest the presence of DNA polymerases encoded by mobile extra-chromosomal genetic elements with uneven distribution among Thermus strains in nature.

To isolate DNA for amplification of polymerase genes, numerous Thermus and Meiothermus strains were used as well as environmental complex biomass samples as described in Example 1. Degenerate PCR methods were used to amplify gene fragments corresponding to polymerase genes as described in Example 2. A number of strains gave amplified gene fragments corresponding to a novel type of DNA polymerase distantly related to Taq DNA polymerase I and other known corresponding polymerases of that type in other Thermus species. Surprisingly, the novel DNA polymerases disclosed here were found to be apparently closest related to Aquifex and Desulfitobacterium DNA polymerases when compared to known DNA polymerases described in the prior art. Still, the similarity to these DNA polymerase is limited (seq. identity about 30-35%) and the polymerases of the invention cannot be considered closely related to even these closest known relatives. In some cases, gene fragments were amplified of both the novel type of polymerase as well as the conventional DNA polymerase I gene already identified in Thermus species. The novel type of DNA polymerase gene was also successfully amplified from environmental samples and a sequence of that origin is disclosed herein as SEQ ID: NO 3. This demonstrates that the genes of the invention can be obtained from different sources apart from isolated Thermus strains. Consequently, it is not excluded that gene fragments of the invention may be obtainable from bacterial strains other than strains belonging to the genus Thermus and may even be of non-bacterial origin such as from bacteriophage genomes.

Partial sequencing of the amplified fragments was carried out as described in Example 3. The sequence of identified polymerase gene fragments were used to design inverse primers for retrieval of whole polymerase genes (Example 4). A novel type of polymerase gene was apparent in only some of strains analysed in the study. Hybridization experiments were carried out to confirm the presence of the new type of polymerase genes in the strains (Example 5). The results indicate that the strains belonging to the species T. aquaticus, T. thermophilus, T. filiformis, “T. eggertssonii”, T. igniterrae and T. flavus do not contain this type of polymerase gene whereas some strains of the species T. scotoductus, T. brockianus, T. oshimai and T. atranikianii contain DNA polymerases of the invention.

Two clones were selected for characterization. Initially, we used a clone expressing the gene designated as Pol-11 (pAP17b) expressed in pJOE3075 vector without His-tail and in E. coli cells BL21-RIL. Later, another Pol-11 clone (pAP18b with vector pJOE3075 in E. coli BL21RIL without DE3) was used as source of the enzyme carrying a His-tail. The gene for Pol-11 originated from T. antranikianii strain 2120. Later in the comparisons another clone with a different gene was used as well. This was designated Pol-3 and was cloned and expressed in pJOE3075 vector with His-tail and in E. coli BL21+DE3. The gene for Pol-3 originated from T. brockianus strain 140. The nucleic acid sequences of the selected DNA polymerase genes are shown as SEQ ID NO: 1 (Pol-11), SEQ ID NO: 2 (Pol-3) and SEQ ID NO: 3 (Pol-62). Example 6 describes how complete genes of the new type of DNA polymerases were cloned into expression vectors, the genes expressed and the corresponding clones tested for activity.

The DNA polymerase Pol-11 polypeptide was chosen as a suitable candidate for the detailed characterization of the type of enzymes disclosed by the invention. Example 7 describes experiments aimed at finding optimal reaction temperature and pH as well as the effects of varying the concentration of some salts. Polymerase Pol-11 had a temperature optimum around 50-55° C. and a pH optimum pH of approximately 8.5. Heat stability and activity at different temperatures of polymerase Pol-11 was studied as described in Example 8 and the effects of varying the template DNA and nucleotides were observed as described in Example 9. DNA polymerase Pol-11 was expressed and purified (Example 10) for further characterization. Pol-11 polymerase was found to have substantial activity at temperatures up to 90° C. Also, after incubation of the enzyme for 15 minutes up to 94° C., the enzyme still showed residual activity which could be further increased by addition of high concentration of L-proline during the incubations. The invention thus pertains to DNA polymerases having strand displacement activity at elevated temperatures such as above 50° C., such as up to 100° C., such as between 50 and 80° C. The resistance of the polypeptides to heat inactivation may permit their use in applications employing elevated temperatures including denaturing conditions such as in the use of PCR. The invention pertains also to the use of these polypeptides with stabilizing agents such as L-proline.

We also cloned and expressed a gene retrieved from a complex biomass sample from a hot spring (Badstofuhver, S-Iceland) at 85° C., pH 8. The DNA material used for amplification of the gene was obtained from an environmental sample containing heterogeneous genetic material from the plurality of microbial species found in the ecosystem at the sampling site. The gene obtained from the complex biomass sample may therefore have originated from an organism not belonging to the genus Thermus. The biomass gene product was designated Pol-62 and was found to be very active with strand displacing activity similar to Pol-11 and Pol-3 (data not shown). We have thus demonstrated that DNA polymerases of the type disclosed by the invention can be obtained from environmental DNA without prior isolation of microbial strains such as Thermus strains.

A number of experiments were carried out to investigate if the novel type of polymerase genes were located on an extrachromosomal genetic element (Example 11). The experiments suggest that novel polymerase genes disclosed by the invention are located on plasmids, or possibly a bacteriophage genome carried by the host, such as a prophage, found in some but not all bacterial strains of the genus Thermus. As an example, an apparent plasmid band was observed in strain 140; isolating it from the agarose gel and digesting with exonucleases (both exo I and exo III) still gave a PCR product of the correct size with the specific primers for the new polymerase gene. The results also indicate that the plasmid is larger than 12 Kb at least in some of the tested strains. Based on these results we suggest that the gene of the new DNA polymerase is located on a plasmid. The properties of these novel DNA polymerases also seem consistent with their possible function in vivo in plasmid replication such as through a rolling-circle replication mechanism (del Solar et al. 1998).

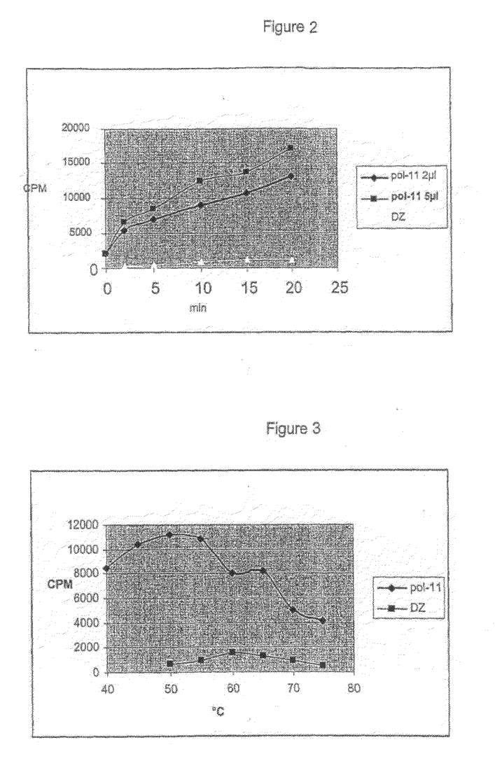

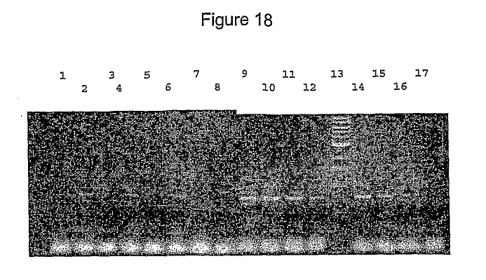

Initial characterization of Pol-11 (see e.g. Example 6) indicated a drastic difference in nucleotide incorporation of Pol-11 and the enzyme used as comparison control which was the Thermus DNA polymerase I enzyme DyNAzyme™ (Finnzymes Oy, Finland) as can for example be seen in FIG. 2. This observation indicated that the enzyme is strand displacing and was continuing incorporation of nucleotides until the nucleotides were practically finished. The very steep incorporation during the first two minutes, and also upon adding template to the reaction, indicates a high rate of the reaction catalyzed by the enzyme (FIG. 18). The observations prompted further characterization of Pol-11 and a comparison with Pol-3 which is from a T. brockianus strain which was also used in previous activity screening of Thermus DNA polymerases (Hjorleifsdottir, 2002). These two enzymes were purified and all further experiments were performed with purified enzymes.

Purified DNA polymerase Pol-11 was compared to DNA polymerase I from “Thermus eggertssonii” (Teg). Teg DNA polymerase (produced by Prokaria Ltd. for internal use) has similar characteristics as Taq DNA polymerase I and corresponds closely to the commercially available enzyme DyNAzyme™. Standard DNA polymerase test was done as described in Example 12. Pol-11 showed good incorporation of labeled nucleotides in contrast to Teg DNA polymerase which gave little incorporation in the test.

Pol-11 was compared to Phi29 DNA polymerase which is the conventional enzyme used for strand displacement amplifications (Example 13). The experiment shows that Pol-11 is needed to initiate primer extension, and that the nature of this extension mimics primer extension of the strand displacing enzyme Phi29. The experiments in Example 13 also demonstrate that amplification using Pol-11 occurs even in absence of primers. This amplification most likely starts from nicked relaxed plasmid DNA (supercoiled plasmid DNA does not act as template). This suggests that Pol-11 is able to start DNA synthesis at a nick (single-strand break in the DNA) in double stranded DNA and is thus a good demonstration of the strand displacement function of the enzyme. In example 14, the requirement of Pol-11 for nucleotides was assessed. The result shows that the enzyme is dependent on all four nucleotides, indicating that the amplification is not an unspecific incorporation of nucleotides but rather that it is directed by the template. Exonuclease activity of Pol-11 was tested as described in Example 15, indicating that the enzyme has exonuclease activity.

The specific activity of Pol-11 was determined (Example 16) and found to be about 360.000 units/mg where each unit is defined as the amount of enzyme required to convert 10 nmol of dNTP to a material in 30 minutes under the conditions described in Example 16. In contrast, the specific activity of Phi29 DNA polymerase, which was also determined in the same experiment for comparison, is only 10.800 units per mg. The conditions for activity determinations were the same for both enzymes in terms of relative amount of template, nucleotides and enzyme. There is a clear difference in the properties of Pol-11 and Phi29 in terms of efficiency in amplification of activated DNA without primers. Pol-11 DNA polymerase has an order of magnitude higher activity than Phi29 DNA polymerase. The polypeptides of the invention can be used to improve existing methods of strand displacement amplification of DNA and extend the range of conditions and type of applications that can be applied. The invention thus provides improved methods for amplification of DNA using the isolated polypeptides of the invention.

Pol-11 can be successfully used to amplify genomic DNA from minute amounts of starting material, such as human genomic DNA, as demonstrated in Example 17. Genomic material amplified by Pol-1, such as human DNA, can be used for specific amplification of a genomic marker such as a gene. Example 18 demonstrates the amplification of the human B-actin gene from human genomic DNA after whole genome amplification using Pol-11. It is possible to clone by PCR a normal Beta-actin gene from material amplified by Pol-11 containing less original template than applicable for PCR. Another experiment was done (Example 19) to verify amplification of human DNA from starting material in amounts less than sufficient for normal PCR amplification using specific primers. After amplification, the amplified material could be used for detectable amplification of specific gene using PCR. Pol-11 can also be used to amplify genomic material from other sources such as salmon DNA. Example 20 describes the amplification of Atlantic salmon genomic DNA. The genomic DNA amplified by Pol-11 can be used in procedures such as genotyping as also illustrated by Example 20. From the results described in Example 20, it can be calculated that the amplification of the starting material corresponds to 200- to 1000-fold amplification. The results also indicate that the amplified material has no loss of allele representation which is consistent with unbiased amplification of the genomic DNA.

As described herein, the inventors have isolated and characterized polypeptides having DNA polymerase strand displacement activity. The polypeptides of the invention shows substantial DNA polymerase strand displacement activity and are by inference substantially stable (i.e. correctly folded and soluble) at temperatures up to about 95° C. Substantially stable means that there is a significant proportion of the polypeptides capable of showing DNA polymerase activity at a particular temperature, such as displaying substantial activity, such as showing more than 10% activity relative activity at optimal temperature, for at least ten minutes at the particular temperature. Substantially stable may also mean that the polypeptides are capable of showing substantial DNA polymerase activity after at least ten minutes prior incubation at a particular temperature. The polypeptides retain at least 20% activity upon incubation for at least 24 hours at temperatures of at least about 60° C., and retain substantial activity at temperatures in the range from about 30° C. to about 95° C. This extended range of thermostability as compared to mesophilic counterparts is useful in various applications known to those skilled in the art and as set forth herein.

### Sequence and Structure-Function Relationships

DNA polymerases are divided in different families including family A and family B and a number of other families. Family A includes DNA polymerase I in bacteria, for example E. coli DNA polymerase I, Taq DNA polymerase I and B. stearothermophilus DNA polymerase I, and also DNA polymerases from other sources such as bacteriophage T7 DNA polymerase. Family B includes many archaeal polymerases and a number of polymerases from bacteriophages, such as bacteriophage Phi29 DNA polymerase and bacteriophage T4 DNA polymerase (Albà 2001; Blanco et al. 1991). Families A and B are characterized by conserved sequence motifs including residues of functional importance such as catalytic residues directly involved in the reaction mechanisms including template directed DNA synthesis and exonuclease activity (Steitz 1999; Brautigam and Steitz 1998). In the polymerase domain of members of family A, three conserved sequence motifs have been identified as being characteristic for this family. The motifs are commonly referred to as motifs A, B and C. Motifs A and C include conserved aspartic residues functioning as ligands to two divalent metal ions in the active site of the polymerase domain which are central to the catalytic mechanism. Motif B in the fingers subdomain contains residues involved in binding incoming dNTPs at the active site (Steitz 1999; Brautigam and Steitz 1998; Albà 2001). As discussed below, this part of the molecule is also involved in the conformational change of the protein during each cycle of nucleotide addition and is directly involved in strand displacement of the downstream non-template DNA strand. For a new polypeptide sequence deduced from a gene isolated from nature and showing significant similarity to family A DNA Polymerases, it is intuitive to conclude, from the overall sequence similarity and the conservation of the sequence motifs including the presence of the identified functionally important residues, that the corresponding polypeptide has DNA polymerase activity.

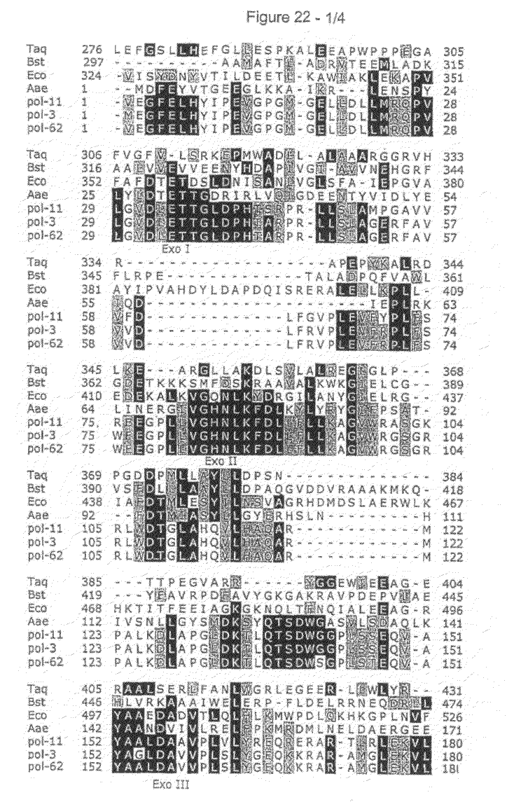

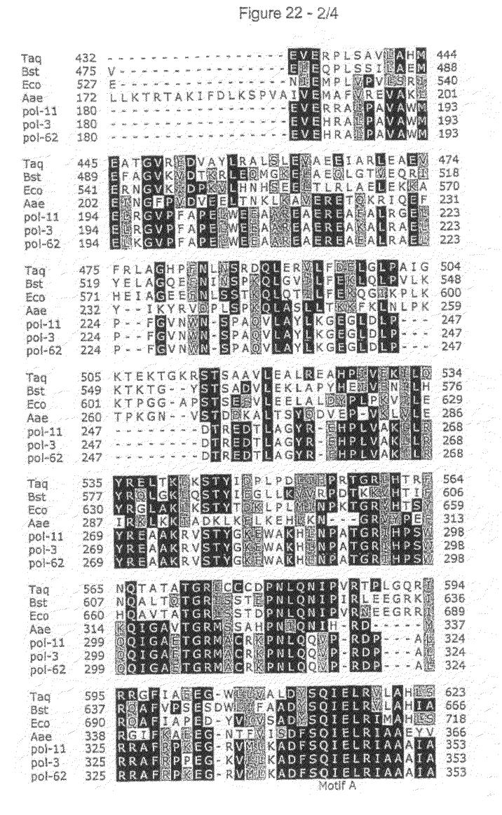

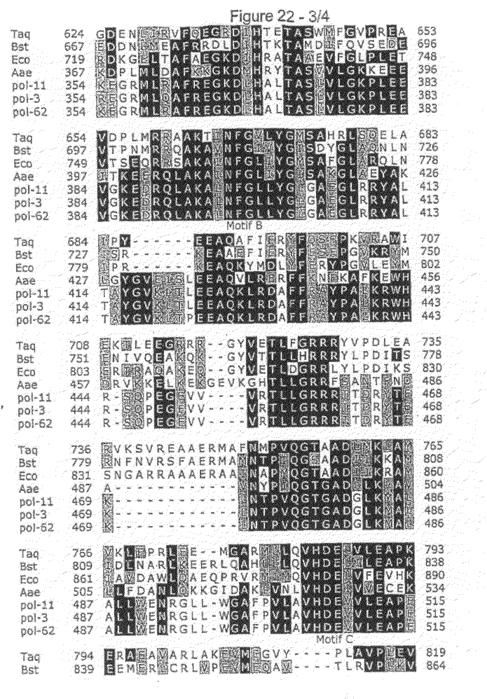

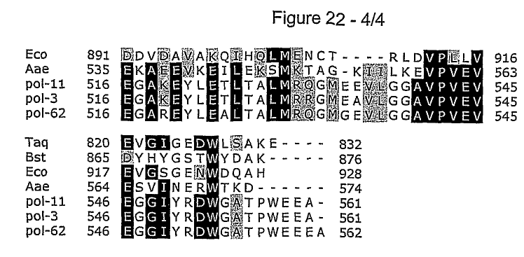

The DNA polymerases of the present invention, exemplified by Pol-11, Pol-3 and Pol-62, belong to family A of DNA polymerases. The characteristic sequence motifs A, B and C are clearly identifiable and show high degree of conservation compared to sequences of other members of the family A (see Example 21). The catalytically important residues, identified in sequence alignment with representative prior art DNA polymerase sequences from other sources (see FIG. 22), include ligands to the metal ions, for example Asp340 of motif A and Asp507 of motif C in pol-11 and other residues at the active site, such as the conserved Arg, Lys and Tyr residues of motif B (Arg389, Lys393 and Tyr401 in Pol-11). Inspection of available structural information, including co-crystal complexes of polymerases and nucleic acids (see Example 21), allows for more careful inspection of the location of residues in the polypeptides of the present invention, with respect to e.g. the template nucleic acids, and thus the functional significance of certain amino residues can be indicated. As illustrated in detail below, certain unique sequence features, in the light of the prior art structural information, are implicated in the unique strand displacement properties of the polypeptides of the present invention.

Although many polymerases of family A contain a 5′ exonuclease domain, some members of the family, including the polypeptides of the present invention, naturally lack this domain and the corresponding activity. On the other hand, a 3′ exonuclease domain is present in the polypeptides of the invention. The catalytic residues of the 3′ exonuclease domain have been well characterized and are found in characteristic sequence motifs (exo I, exo II and exo III, Blanco et al. 1991). Acidic residues of the conserved sequence motifs function as ligands to metal ions in the active site of the exonuclease domain (Brautigham and Steitz 1998; Steitz and Steitz 1993). Some DNA polymerases, such as Bst DNA polymerase, naturally lack this activity as indicated by substitutions at the corresponding active site residues (Aliotta et al. 1996; Kiefer et al. 1997). The polypeptides of the present invention contain the catalytic residues of the 3′ exonuclease domain indicating that these enzymes have exonuclease activity as also confirmed by experiments (Example 15). In Pol-11 for example, catalytic residues implicated in 3′ exonuclease activity are the acidic residues Asp32, Glu34 and Asp89 which form the ligands to divalent metal ions required for the catalytic mechanism of the 3′ exonuclease domain (Freemont et al. 1988). The polypeptides of the invention thus most likely are proofreading DNA polymerases. In contrast, Bst DNA polymerase I and Taq DNA polymerase lacks those sequence features and has been shown to lack proofreading 3′ exonuclease activity (Aliotta et al. 1996).

The specific features of the polypeptides of the invention can be used to distinguish them from prior art DNA polymerases, including those used for strand displacement amplification methods, by their features. As shown in Table 1, the polypeptides of the invention have a unique combination of functionally important features. Accordingly, the invention pertains to DNA polymerases belonging to family A DNA polymerases, having a functional 3′ exonuclease domain (proofreading activity), and preferably lacking a 5′ exonuclease domain, having very substantial strand displacement activity and being thermophilic. In addition, the polypeptides of the invention also have unique structural features which are linked to their exceptional strand displacement properties as discussed below.

TABLE 1
Properties of representative DNA polymerases

| | | 3′ exo | 5′ exo | Strand | Thermo- |
|---|---|---|---|---|---|
| Identity | Family | activity | activity | displacement | philic |
| Pol-11, Pol-3, | A | yes | no | yes | yes |
| Pol-62 | | | | | |
| Phi29 DNA pol | B | yes | no | yes | no |
| Taq DNA pol | A | no | yes | no | yes |
| Bst DNA pol | A | no | yes | yes | yes |
| Vent DNA pol* | B | yes | no | yes | yes |
| Aquifex DNA pol | A | yes | no | no | yes |

*Vent DNA pol is a commercial archaeal polymerase

Bst DNA polymerase and the polymerases of the invention have several common features but important functional differences as well. Bst DNA polymerase is of the same family (family A) as the DNA polymerases of the invention and it is also active at elevated temperatures such as around 50° C. Bst DNA polymerase from which the 5′ exonuclease domain has been excised (“Large fragment”) has also high strand displacing activity (WO 97/39113). The large fragment of Bst DNA polymerases is composed of the same basic domains as the polypeptides of the invention, i.e. a 3′ exonuclease-like domain and polymerase domain (Kiefer et al. 1997), and it is lacks the 5′ exonuclease domain since it has been artificially removed. The polypeptides of the invention however naturally lack a 5′ exonuclease domain and therefore are naturally and probably better adapted to function in the absence of a 5′ exonuclease domain than the large fragment of Bst DNA polymerase. The Bst DNA polymerase is also substantially less thermostable, compared to the DNA polymerases of the present invention, since it reportedly can be inactivated by 15 min incubation at 75° C. (Epicentre technical sheet) or 10 min at 80° C. (New England Biolabs technical sheet). More importantly, the Bst DNA polymerase is lacking a functional 3′ exonuclease domain (Aliotta et al. 1996) which in contrast is functional in the polypeptides of the invention. This is a very distinctive difference and the consequent lack of proof reading activity in Bst DNA polymerase is a disadvantage for its general use in amplification reaction due to high error rate. This may include for example single base pair errors but due to the nature of the strand displacement reaction, there seems to be also a great risk of other errors in absence of proof reading activity such as chimer formation due to unspecific priming events. These shortcoming of Bst DNA polymerase have been noted in the prior art and it has been suggested that the use of this enzyme should preferably be used in applications which are not sensitive to single-base errors such as involving hydbridizations (Lage et al. 2003).

The Phi29 DNA polymerase seems to be the most commonly used DNA polymerase for strand displacement applications and is considered to be the best suited enzyme for these applications (Technical reference sheet, New England Biolabs), i.e. Phi29 DNA polymerase is considered the current industry standard. The polypeptides of the invention are however distinctively different from Phi29 DNA polymerase, they belong to a different family and are thermostable with optimal activity at temperatures above about 50° C. whereas Phi29 DNA polymerase has optimal activity around 30° C. The range of applications which can be employed is therefore different for the different types of enzymes. For example, strand displacement amplification of DNA in absence of primers may be favored by higher temperatures by increasing rate of new priming events through pairing of identical regions in DNA strands made in the initial phase of amplification (gap filling and strand displacement replication of initial template). We propose that at elevated temperatures (e.g. above about 50° C.) strand displacement is facilitated as less energy is needed for strand separation but strand displacement becomes inhibited at even higher temperatures due to fewer priming events. The temperature range wherein the DNA polymerases of the invention show high activity, such as between 40 and 70° C., may include optimum temperature for many strand replacement amplification reactions. We have demonstrated that the specific activity of the DNA polymerases of the invention, measured as amplification of activated DNA, greatly exceeds the specific activity of Phi29 DNA polymerase.

Structural studies of various DNA and RNA polymerases and their complexes with nucleotides, template and primer oligonucleotides have provided great insight into various aspects of the mechanisms of these enzymes. This includes structure-function relationship with respect to properties such as replication mechanism, fidelity of synthesis, nuclease activity, processivity and strand displacement. Structural determinations of representatives from these families have revealed the differences and similarities in various structural features of members of different families as well as within families. For example, the palm in the polymerase domain seems quite similar in different families whereas the fingers and thumb regions are more different (Steitz 1999; Brautigam and Steitz 1998; Beard and Wilson 2003; Albà 2001).

The fingers regions of the polymerase domain have been shown to undergo conformation changes related to binding and hydrolysis of incoming nucleoside triphosphate and thus play important part in fidelity of synthesis as well as translocation of the polymerase along the template and displacement of downstream non-template nucleic acid strand. A correct Watson-Crick basepair between template and incoming nucleotide at the polymerase active site will facilitate the conformational change of the fingers domain, which is essential for catalysis of the reaction, whereas a non-Watson-Crick basepair will hinder the conformational change thereby stalling the reaction. Any incorrect nucleotide incorporation will destabilize the formed duplex and increase rate of excision of the incorrect nucleotide by movement of the strand to the active site of the 3′ proofreading exonuclease. Furthermore, it has been shown that conformational change of the fingers domain is essential for translocation and strand displacement. The movement of the fingers region demonstrated by different co-crystal structures of T7 RNA polymerase, a strand displacing polymerase and a homologue of DNA polymerase I, seems to be driven by the binding of the incoming nucleotide and subsequent release of the pyrophosphate leading to closure and opening of the fingers domain. The conformational change of the fingers domain can be describes as rotation about a pivot point leading to 3.4 Å movement of the product duplex which is the required relative translocation of the polymerase along template during a single cycle of nucleotide addition. As one of the helices of the fingers domain is situated between the template and non-template strands, the movement of the fingers will lead to displacement of the downstream non-template strand (Steitz and Yin 2003, Yin and Steitz 2004, see also complementing material in Yin and Steitz 2004). The fingers region has also been suggested to contain the structural determinants of strand displacement in Human Immunodeficiency Virus I reverse transcriptase (Fisher et al. 2003). The polypeptide region, in close proximity to the site of opening of the downstream duplex, in RNA polymerase and E. coli DNA polymerase I are structurally similar and the corresponding structures were superimposed as described in Example 21 guided by the work of Yin and Steitz (Yin and Steitz 2004). This region corresponds to the sequence around Glu406 in Motif B in the preferred polypeptides of the invention represented by Pol-11, Pol-3 and Pol-62 (FIG. 22). As can be seen from the structural superposition, the residue closest to the first hydrogen bonded base pair in the downstream duplex is a phenylalanine in both RNA polymerase and E. coli DNA polymerase I. However, there is a clear difference between the two structures in the following loop where the RNA polymerase has a more extensive loop region forming a platform with and extra point of attachment for the outgoing displaced DNA strand with bonds between the DNA strands and some of the amino acid residues in the loop. Interestingly, as can be seen from the alignment of the sequences in FIG. 22, the corresponding loop in the polypeptides of the invention is more extensive than in DNA polymerase I from Thermus and in that sense resembles more the RNA polymerase loop. Similarly, the polypeptides of the present invention could thus provide analogous platform for attachment of the displaced strand and thereby facilitate strand displacement. In the polypeptide region close to the displaced strand, including the extended loop, are for example basic residues and an aromatic tyrosine which could be appropriate for stabilizing the displaced strand. Furthermore, it can be seen from the sequence alignment and the structural superpositions, that the residue in the polypeptides of the invention, which is closest to the first base pair of the downstream duplex, is not a phenylalanine residue by rather a negatively charged glutamate residue (Glu406). This particular amino acid residue could act repelling to the negatively charged sugar-phosphate backbone of the displaced strand and thus facilitate breaking of the hydrogen bonds of the base pair to be disrupted during each cycle.

From the observations discussed above, it seems likely that a particular region in the polypeptides of the invention is important and plays a direct part in the displacement mechanism and may be crucial for the high efficiency of strand displacement seen in these enzymes. More specifically, this refers to the regions of residues Glu406 to Leu422 in Pol-11 polypeptide and corresponding regions in Pol-3 and Pol-62 (see FIG. 22), partly overlapping motif B in the fingers domain. This region is well conserved in all three polypeptides and consists of the amino acid sequence 406EGLRRYALTAYGVKLTL422 in Pol-11 and Pol-3 (one substitution in Pol-62 which has Pro at position 422). Interestingly, this region of the sequence is partly forming an insert in a sequence alignment compared to Taq DNA polymerase, Bst DNA polymerase I and DNA polymerase I from E. coli as well as most members of family A as can be seen with reference to protein family databases such as Pfam (DNA polymerase family A, accession number PF00476). The position of the insert is unlikely to be very misplaced in the alignment in FIG. 22 as rather good similarity is seen on both sides of this region (more accurate structure-based alignment, if structural information of polypeptides of the invention were available, would perhaps shift the insert slightly such as 1 or a few positions to the left). As discussed above the specific sequence and length of this region, in particular residues 406 to 422, is likely to be important for the strand displacement activity of the polypeptides of the invention. The insert most likely corresponds to an extended loop (compared to most DNA polymerases of family A) located close to the site of strand displacement when the polypeptides of the invention are bound to template DNA with a downstream DNA duplex of template and non-template strands. The residue closest to the downstream duplex basepair to be disrupted during each cycle of conformational change of the fingers is Glu406 in the polypeptides of the invention.

To our knowledge, the strand displacement properties of Aquifex aeolicus DNA polymerase I have not been reported and we take that as an indication that the enzyme does not have high strand displacing activity. Although Aquifex aeolicus DNA polymerase I has an insert of comparable size, the sequence is significantly different compared to the sequences of the polypeptides of the present invention with only 8 identical residues out of 17. Importantly, the residues implied here as functionally significant in strand displacement are of very different character in Aquifex aeolicus DNA polymerase compared to the DNA polymerases of the invention, with for example charged residues replaced by residues of opposite charge. Thus, the crucial residue located at the site of base pair opening, which is a glutamate residue (Glu406) in the polypeptides of the invention, is a lysine residue in Aquifex aeolicus DNA polymerase I.

We infer from all the evidence presented here that we have discovered a novel group of DNA polymerases. Not only are their structural and functional features distinct but also they form a distinct phylogenetic branch. Moreover, the polymerases of the invention do not seem to be a part of the common house-holding enzymes present throughout the bacterial kingdom but appear to be rather required in special circumstances and are encoded by genes not normally found in bacterial genomes but rather in the genetic make-up of only certain organisms in nature.

### Nucleic Acids of the Invention

One aspect of the invention pertains to isolated nucleic acid sequences, encoding polypeptides having DNA polymerase strand displacement activity, as described above. Sequences of preferred isolated nucleic acids of the invention are included herein as SEQ ID NO: 1, SEQ ID NO: 2 and SEQ ID NO: 3.

The nucleic acid molecules of the invention can be DNA, or can also be RNA, for example, mRNA. DNA molecules can be double-stranded or single-stranded; single stranded RNA or DNA can be the coding, or sense, strand or the non-coding, or antisense, strand. Preferably, the nucleic acid molecule comprises at least about 100 nucleotides, more preferably at least about 150 nucleotides, and even more preferably at least about 200 nucleotides. In one embodiment the nucleic acid of the invention comprises a sequence which encodes at least a fragment of the amino acid sequence of a polypeptide of the invention; alternatively, the nucleotide sequence can include at least a fragment of a coding sequence along with additional non-coding sequences such as non-coding 3′ and 5′ sequences (including regulatory sequences, for example).

Additionally, the nucleotide sequence(s) can be fused to a marker sequence, for example, a sequence which encodes a polypeptide to assist in isolation or purification of the polypeptide. Representative sequences include, but are not limited to, those which encode a glutathione-S-transferase (GST) fusion protein or a histidine tag. In one embodiment, the nucleotide sequence contains a single ORF in its entirety (e.g., encoding a polypeptide, as described below); or contains a nucleotide sequence encoding an active derivative or active fragment of the polypeptide; or encodes a polypeptide which has substantial sequence identity to the polypeptides described herein.

The nucleic acid molecule of the invention can be fused to other coding or regulatory sequences. Thus, recombinant DNA contained in a vector is included in the definition of “isolated” as used herein. Also, isolated nucleic acid molecules include recombinant DNA molecules in heterologous host cells, as well as partially or substantially purified DNA molecules in solution. “Isolated” nucleic acid molecules also encompass in vivo and in vitro RNA transcripts of the DNA molecules of the present invention. An isolated nucleic acid molecule or nucleotide sequence can include a nucleic acid molecule or nucleotide sequence which is synthesized chemically or by recombinant means. Therefore, recombinant DNA contained in a vector is included in the definition of “isolated” as used herein. Also, isolated nucleotide sequences include recombinant DNA molecules in heterologous organisms, as well as partially or substantially purified DNA molecules in solution. In vivo and in vitro RNA transcripts of the DNA molecules of the present invention are also encompassed by “isolated” nucleotide sequences. Such isolated nucleotide sequences are useful in the manufacture of the encoded polypeptide, as probes for isolating homologous sequences, for gene mapping or for detecting expression of the gene, such as by Northern blot analysis.

The present invention also pertains to nucleotide sequences which are not necessarily found in nature but which encode the polypeptides of the invention. Thus, DNA molecules which comprise a sequence which is different from the naturally occurring nucleotide sequence but which, due to the degeneracy of the genetic code, encode polypeptides of the present invention are also subject of this invention. The invention also encompasses variations of the nucleotide sequences of the invention, such as those encoding active fragments or active derivatives of the polypeptides as described below. Such variations can be naturally occurring, or non-naturally occurring, such as those induced by various mutagens and mutagenic processes. Intended variations include, but are not limited to, addition, deletion and substitution of one or more nucleotides which can result in conservative or non-conservative amino acid changes, including additions and deletions. Preferably, the nucleotide or amino acid variations are silent or conservative; that is, they do not alter the characteristics (e.g. structure, flexibility and electrostatic microenvironment within the protein) or activity of the encoded polypeptide. However, variations may alter the various properties of the polypeptides encoded by the nucleic acids while preferably still retaining substantial enzyme activity.

The invention described herein also relates to fragments of the isolated nucleic acid molecules described herein. The term “fragment” is intended to encompass a portion of a nucleotide sequence described herein which is from at least about 15 contiguous nucleotides to at least about 50 contiguous nucleotides or longer in length; such fragments are useful as probes and also as primers. Particularly preferred primers and probes selectively hybridize to the nucleic acid molecule encoding the polypeptides described herein. For example, fragments which encode polypeptides that retain enzyme activity, as described below, are particularly useful.

Other alterations of the nucleic acid molecules of the invention can include, for example, labeling, methylation, internucleotide modifications such as uncharged linkages (e.g., methyl phosphonates, phosphotriesters, phosphoamidates, carbamates), charged linkages (e.g., phosphorothioates, phosphorodithioates), pendent moieties (e.g., polypeptides), intercalators (e.g., acridine, psoralen), chelators, alkylators, and modified linkages (e.g., alpha anomeric nucleic acids). Also included are synthetic molecules that mimic nucleic acid molecules in the ability to bind to a designated sequence via hydrogen bonding and other chemical interactions. Such molecules include, for example, those in which peptide linkages substitute for phosphate linkages in the backbone of the molecule (polypeptide nucleic acids, as described in Nielsen, et al., 1991)).

The invention also encompasses nucleic acid molecules which hybridize under high stringency hybridization conditions, such as for selective hybridization, to a nucleotide sequence described herein (e.g., nucleic acid molecules which specifically hybridize to a nucleotide sequence encoding polypeptides described herein, and, optionally, have an activity of the polypeptide). Hybridization probes are oligonucleotides which bind in a base-specific manner to a complementary strand of nucleic acid.

Such nucleic acid molecules can be detected and/or isolated by specific hybridization (e.g., under high stringency conditions). “Stringency conditions” for hybridization is a term of art which refers to the incubation and wash conditions, e.g., conditions of temperature and buffer concentration, which permit hybridization of a particular nucleic acid to a second nucleic acid; the first nucleic acid may be perfectly (i.e., 100%) complementary to the second, or the first and second may share some degree of complementarity which is less than perfect (e.g., 60%, 75%, 85%, 95%). For example, certain high stringency conditions can be used which distinguish perfectly complementary nucleic acids from those of less complementarity.

“High stringency conditions”, “moderate stringency conditions” and “low stringency conditions” for nucleic acid hybridizations are explained on pages 2.10.1-2.10.16 and pages 6.3.1-6 in Current Protocols in Molecular Biology (Ausubel, F. M. et al., “Current Protocols in Molecular Biology”, John Wiley & Sons, (2001)) the teachings of which are hereby incorporated by reference. The exact conditions which determine the stringency of hybridization depend not only on ionic strength (e.g., 0.2×SSC, 0.1×SSC), temperature (e.g., room temperature, 42° C., 68° C.) and the concentration of destabilizing agents such as formamide or denaturing agents such as SDS, but also on factors such as the length of the nucleic acid sequence, base composition, percentage mismatch between hybridizing sequences and the frequency of occurrence of subsets of that sequence within other non-identical sequences. Thus, high, moderate or low stringency conditions can be determined empirically.

By varying hybridization conditions from a level of stringency at which no hybridization occurs to a level at which hybridization is first observed, conditions which will allow a given sequence to hybridize (e.g., selectively) with the most similar sequences in the sample can be determined. Exemplary conditions are described in Krause, M. H. and S. A. Aaronson, Methods in Enzymology, 200:546-556 (1991). Also, in, Ausubel, et al., “Current Protocols in Molecular Biology,” John Wiley & Sons (2001), which describes the determination of washing conditions for moderate or low stringency conditions. Washing is the step in which conditions are usually set so as to determine a minimum level of complementarity of the hybrids. Generally, starting from the lowest temperature at which only homologous hybridization occurs, each degree C. by which the final wash temperature is reduced (holding SSC concentration constant) allows an increase by 1% in the maximum extent of mismatching among the sequences that hybridize. Generally, doubling the concentration of SSC results in an increase in Tₘ of 17° C. Using these guidelines, the washing temperature can be determined empirically for high, moderate or low stringency, depending on the level of mismatch sought.

For example, a low stringency wash can comprise washing in a solution containing 0.2×SSC/0.1% SDS for 10 minutes at room temperature; a moderate stringency wash can comprise washing in a pre-warmed solution (42° C.) solution containing 0.2×SSC/0.1% SDS for 15 min at 42° C.; and a high stringency wash can comprise washing in prewarmed (68° C.) solution containing 0.1×SSC/0.1% SDS for 15 min at 68° C. Furthermore, washes can be performed repeatedly or sequentially to obtain a desired result as known in the art.

Equivalent conditions can be determined by varying one or more of the parameters given as an example, as known in the art, while maintaining a similar degree of identity or similarity between the target nucleic acid molecule and the primer or probe used.

Hybridizable nucleic acid molecules are useful as probes and primers, e.g., for diagnostic applications. Such hybridizable nucleotide sequences are useful as probes and primers for diagnostic applications. As used herein, the term “primer” refers to a single-stranded oligonucleotide which acts as a point of initiation of template-directed DNA synthesis under appropriate conditions (e.g., in the presence of four different nucleoside triphosphates and an agent for polymerization, such as, DNA or RNA polymerase or reverse transcriptase) in an appropriate buffer and at a suitable temperature. The appropriate length of a primer depends on the intended use of the primer, but typically ranges from 15 to 30 nucleotides. Short primer molecules generally require cooler temperatures to form sufficiently stable hybrid complexes with the template. A primer need not reflect the exact sequence of the template, but must be sufficiently complementary to hybridize with a template. The term “primer site” refers to the area of the target DNA to which a primer hybridizes. The term “primer pair” refers to a set of primers including a 5′ (upstream) primer that hybridizes with the 5′ end of the DNA sequence to be amplified and a 3′ (downstream) primer that hybridizes with the complement of the 3′ end of the sequence to be amplified.

The invention also pertains to nucleotide sequences which have a substantial identity with the nucleotide sequences described herein; particularly preferred are nucleotide sequences which have at least about 10%, preferably at least about 20%, more preferably at least about 30%, more preferably at least about 40%, even more preferably at least about 50%, yet more preferably at least about 70%, still more preferably at least about 80%, and even more preferably at least about 90% identity, and still more preferably 95% identity, with nucleotide sequences described herein. Particularly preferred in this instance are nucleotide sequences encoding polypeptides having DNA polymerase strand displacement activity as described herein.

To determine the percent identity of two nucleotide sequences, the sequences are aligned for optimal comparison purposes (e.g., gaps can be introduced in the sequence of a first nucleotide sequence). The nucleotides at corresponding nucleotide positions are then compared. When a position in the first sequence is occupied by the same nucleotide as the corresponding position in the second sequence, then the molecules are identical at that position. The determination of percent identity or similarity scores between two sequences can be accomplished using a mathematical algorithm. A preferred, non-limiting example of a mathematical algorithm utilized for the comparison of two sequences is the algorithm of Karlin, et al., Proc. Natl. Acad. Sci. USA, 90:5873-5877 (1993). Such an algorithm is incorporated into the BLAST programs (e.g. BLASTN for nucleotide sequences or BLASTP for protein sequences) which can be used to identify sequences with high similarity scores to nucleotide or protein sequences of the invention. To obtain gapped alignments for comparison purposes, Gapped BLAST can be utilized as described in Altschul et al., Nucleic Acids Res, 25:3389-3402 (1997). When utilizing BLAST and Gapped BLAST programs, the default parameters of the respective programs (e.g., BLASTN) can be used. See the BLAST programs provided by National Center for Biotechnology Information, National Library of Medicine, National Institutes of Health. In one embodiment, parameters for sequence comparison can be set at W=12. Parameters can also be varied (e.g., W=5 or W=20). The value “W” determines how many continuous nucleotides must be identical for the program to identify two sequences as containing regions of identity. Alignment of sequences and calculation of sequence identity may also be done using for example the Needleman and Wunsch global alignment algorithm (Needleman and Wunsch 1970) useful for both protein and DNA alignments and discussed further below.

The invention also provides expression vectors containing a nucleic acid sequence encoding a polypeptide described herein (or an active derivative or fragment thereof), operably linked to at least one regulatory sequence. Many expression vectors are commercially available, and other suitable vectors can be readily prepared by the skilled artisan. “Operably linked” is intended to mean that the nucleotide sequence is linked to a regulatory sequence in a manner which allows expression of the nucleic acid sequence. Regulatory sequences are art-recognized and are selected to produce the polypeptide or active derivative or fragment thereof. Accordingly, the term “regulatory sequence” includes promoters, enhancers, and other expression control elements which are described in Goeddel, Gene Expression Technology: Methods in Enzymology 185, Academic Press, San Diego, Calif. (1990). For example, the native regulatory sequences or regulatory sequences native to organism can be employed. It should be understood that the design of the expression vector may depend on such factors as the choice of the host cell to be transformed and/or the type of polypeptide desired to be expressed. For instance, the polypeptides of the present invention can be produced by ligating the cloned gene, or a portion thereof, into a vector suitable for expression in an appropriate host cell (see, for example, Broach, et al., Experimental Manipulation of Gene Expression, ed. M. Inouye (Academic Press, 1983) p. 83; Molecular Cloning: A Laboratory Manual, 2nd Ed., ed. Sambrook et al. (Cold Spring Harbor Laboratory Press, 1989) Chapters 16 and 17). Typically, expression constructs will contain one or more selectable markers, including, but not limited to, the gene that encodes dihydrofolate reductase and the genes that confer resistance to neomycin, tetracycline, ampicillin, chloramphenicol, kanamycin and streptomycin resistance. Thus, prokaryotic and eukaryotic host cells transformed by the described expression vectors are also provided by this invention. For instance, cells which can be transformed with the vectors of the present invention include, but are not limited to, bacterial cells such as Thermus scotoductus, Thermus thermophilus, E. coli (e.g., E. coli K12 strains), Streptomyces, Pseudomonas, Bacillus, Serratia marcescens and Salmonella typhimurium. The host cells can be transformed by the described vectors by various methods (e.g., electroporation, transfection using calcium chloride, rubidium chloride, calcium phosphate, DEAE-dextran, or other substances; microprojectile bombardment; lipofection, infection where the vector is an infectious agent such as a retroviral genome, and other methods), depending on the type of cellular host. The nucleic acid molecules of the present invention can be produced, for example, by replication in such a host cell, as described above. Alternatively, the nucleic acid molecules can also be produced by chemical synthesis.

The isolated nucleic acid molecules and vectors of the invention are useful in the manufacture of the encoded polypeptide, as probes for isolating homologous sequences (e.g., from other species), as well as for detecting the presence of a DNA construct comprising a nucleic acid sequence of the invention in a culture of host cells.

This invention, in addition to the isolated nucleic acid molecules encoding an DNA polymerases of the invention, disclosed in SEQ ID NO: 1, SEQ ID NO: 2 and SEQ ID NO: 3, also pertains to substantially similar sequences. Isolated nucleic acid sequences are substantially similar if: (i) they are capable of hybridizing under stringent conditions as described to any of the nucleic acids shown as SEQ ID NO: 1, SEQ ID NO: 2 or SEQ ID NO: 3 or (ii) they encode DNA sequences which are degenerate to any of SEQ ID NO: 1, SEQ ID NO: 2 or SEQ ID NO: 3.

Degenerate DNA sequences encode the amino acid sequence of SEQ ID NO: 4, SEQ ID NO: 5 or SEQ ID NO: 6, but have variations in the nucleotide coding sequences. The invention also relates to nucleotide sequences that are substantially similar and that can be identified by hybridization or by sequence comparison. One means for isolating a nucleic acid molecule encoding a polymerase enzyme is to probe a genomic gene library with a natural or artificially designed probe using art recognized procedures (see, for example: Current Protocols in Molecular Biology, Ausubel F. M. et al. (Eds.) Green Publishing Company Assoc. and John Wiley Interscience, New York, 1989, 1992). It is appreciated to one skilled in the art that for example SEQ ID NO: 1, or fragments thereof (comprising at least 15 contiguous nucleotides), is a particularly useful probe. Other particular useful probes for this purpose are hybridizable fragments to the sequences of SEQ ID NO: 1 (i.e., comprising at least 15 contiguous nucleotides).

It is also appreciated that such probes can be and are preferably labeled with an analytically detectable reagent to facilitate identification of the probe. Useful reagents include but are not limited to radioactivity, fluorescent dyes or enzymes capable of catalyzing the formation of a detectable product. The probes are thus useful to isolate complementary copies of DNA from other animal sources or to screen such sources for related sequences.

### Polypeptides of the Invention

As mentioned above the invention provides in a first aspect an isolated thermostable polypeptide belonging to the DNA polymerase A family which is encoded by a gene sequence obtainable from a Thermus sp. with a molecular weight in the range of about 58-68 kDa. The invention additionally relates to isolated polypeptides having DNA polymerase strand displacement activity.

For the purpose of the present invention, “polypeptides having DNA polymerase strand displacement activity” are defined as polypeptides having DNA polymerase strand displacement activity which catalyze DNA synthesis by addition of deoxynucleotides to the 3′ end of a polynucleotide chain, using a complementary nucleic acid strand as a template and being able to displace an intervening strand of nucleic acid hybridized to the template strand. Strand displacement thus refers to the dissociation of a nucleic acid strand from its nucleic acid template in a 5′ to 3′ direction due to template-directed nucleic acid synthesis by the DNA polymerase. DNA polymerase strand displacement activity is suitable assayed by measuring the incorporation of labeled nucleotide such as described for example by Dean et al. (2002).

As described in the Examples, the applicants have cloned three genes and expressed and characterized the corresponding recombinant polypeptides having DNA polymerase strand displacement activity, which represent preferred embodiments of the invention.

The present invention relates to isolated polypeptides having substantial DNA polymerase strand displacement activity at elevated temperatures, such as above 55° C., and active derivatives or fragments thereof. The invention encompasses the polypeptides having the amino acid sequences shown as SEQ ID NO: 4, SEQ ID NO: 5 and SEQ ID NO: 6 and polypeptides having strand displacement activity with substantially similar amino acid sequences to the sequence as shown in SEQ ID NO: 4, SEQ ID NO: 5 and SEQ ID NO: 6 or derivatives or fragments thereof. Compared to prior art polymerases, the polymerases of the invention are more thermostable, i.e. they retain a significant portion of their activity at higher temperatures such as temperatures above about 70° C. or higher such as temperatures above about 75° C. or 80° C. and preferably above about 90° C., e.g. at temperatures in the range of about 55-95° C. such as in the range of 75-95° C. Preferably, the polymerases of the invention retain at least 10% and more preferably at least 15% or at least 20% of their optimal activity at any of the above mentioned temperatures or temperature ranges, when assayed at such temperature. In useful embodiments the polymerase has at least about 10% of optimum activity when assayed at a temperature of 90° C.

The polymerases of the invention also have significant temperature stability, i.e. they preferably retain substantial activity such at least about 10% or at least 15% or more preferable at least 20% after incubation for a period of time such as at least 15 min or at least 20 or after at least 30 min at a high temperature, such as above about 70° C. or 75° C. or at even higher temperatures such as above about 90 or 95° C., prior to being assayed for activity.

It follows that the DNA polymerase polypeptide of the invention preferably has optimal DNA polymerase strand displacing activity at an elevated temperature such as in the temperature range of about 50-95° C., preferably above about 50° C. and more preferably above about 60° C. and yet more preferably above 70° C. or at a temperature in the range of about 50-60° C. such as about 55° C.

Typically, the polymerase of the invention is a member of family A DNA polymerases as described further hereinabove and in great detail in Steitz T. A. (1999). Additionally, the polymerases of the invention preferably naturally lack a 5′-exonuclease domain, e.g. when isolated from natural sources or after cloning and overexpression of the polymerase of the invention from a suitable host cell.

It will be appreciated that preferred embodiments of the invention provide polymerases comprising a functional 3′ exonuclease domain conferring proof-reading activity to the polymerase which thus has a significantly higher fidelity than prior art polymerases lacking a 3′ exonuclease domain, such as e.g. the BSt polymerase discussed above.

As discussed in detail herein, sequence comparisons and sequence/structure alignment of candidate polymerases of the invention with related polymerases show interesting differences between the polymerases of the invention and prior art enzymes. These features are believed to confer to the polymerases of the invention some of the functional advantages disclosed herein.

Accordingly, the polymerases of the invention preferably have substantial sequence identity to the sequences shown as SEQ ID NO: 4, SEQ ID NO: 5, SEQ ID NO: 6, and in particular, the polymerases preferably have substantial identity in the region referred to as the B motif. In some embodiments the present DNA polymerases having strand displacement activity comprise amino acid sequences aligning to the region between and including residues Glu406 and Leu422 in Pol-11 with comparable length (+/−2 residues) and with at least 60% sequence identity to this particular region and more preferably at least 70% or 80% identity with said region and preferably at least 90% identity to said region and more preferably 95% identity to said region, e.g. identical to said region. Residues that are believed to be particularly important are Asp or Glu aligning with Asp406 in Pol-11 and Arg or Lys aligning with Arg409 and Arg410 of Pol-11 (SEQ ID NO: 4). Accordingly, preferred polymerases of the invention have the sequence D/E-x-x-R/K-R/K aligning with residues 406-410 of SEQ ID NO: 4 and preferably having the sequence E-x-x-R-R, where x refer to any amino acid. In some embodiments the polymerase of the invention has the sequence N/Q-F-G-x-x-Y-G-x-x-x-D/E-x-x-R/K-R/K-x-x-x-x-x-x-x-x-K/R in the region referred to as the B-motif, e.g. positioned in a region which aligns with residues 396-419 of motif B of Pol-11 (SEQ ID NO: 4) and preferably the sequence is N/Q-F-G-x-x-Y-G-x-x-x-E-x-x-R-R-x-x-x-x-x-x-x-x-K. Said sequence is in some embodiments N/Q-F-G-x-x-Y-G-x-x-x-D/E-x-x-R/K-R/K-Y-x-x-x-x-Y-G-x-K/R-I/L/V-S/T in said region, aligning to residues 396-421 of Pol-11. In particular embodiments the sequence in this region is N/Q-F-G-x-x-Y-G-x-x-x-D/E-G-I/L/V-R/K-R/K-Y-A-I/L/V-T/S-x-Y-G-V-K/R-I/L/V-T/S such as N-F-G-L-L-Y-G-L-G-A-E-G-L-R-R-Y-A-L-T-A-Y-G-V-K-I/L-T/S.

In one aspect, the polymerase of the present invention belongs to family A DNA polymerases and has a molecular weight of about 61-65 kDa, preferably the molecular weight is around 63 kDa as measured by SDS-PAGE gel electrophoresis or as inferred molecular weight from the nucleotide sequence of the gene, said molecular weight being the weight of the full-length protein encoded by the naturally-occurring full-length gene. A preferred embodiment of the invention is an isolated polypeptide having DNA polymerase activity, obtained from bacteria of the genus Thermus and having an estimated molecular weight of 61-65 kDa and belonging to family A DNA polymerases. In another aspect the polymerase of the present invention is an isolated polypeptide having DNA polymerase activity encoded by a gene obtained from bacteria of the genus Thermus, said full-length gene encoding a polypeptide having an estimated molecular weight of 61-65 kDa and belonging to family A DNA polymerases.

The DNA polymerases of the present invention contain family A DNA polymerase sequence motifs such as “motif A” in the polymerase domain or the “exo I” motif in the 3′ exonuclease domain as seen in FIG. 22. The structural details in the regions of the conserved sequence motifs set the polymerases of the invention apart from other polymerase, for example in the region of motif A, in the polymerase domain, the polymerases of the invention have the unique sequence L-K-A-D-F-S-Q-I-E-L-R-I-A-A-A and in the region of “exo I” motif, in the exonuclease domain, the polymerases of the invention have the unique sequence L-G-V-D-L-E-T-T-G-L-D-P-H.

In one aspect the invention relates to an isolated polypeptide having DNA polymerase activity comprising a C-terminal polymerase domain having a polymerase active site sequence motif L-K-A-D-F-S-Q-I-E-L-R-I-A-A-A in a region of the polypeptide wherein the residues align with residues 337-351 of SEQ ID NO: 4, when the sequence of said polypeptide is aligned with the sequence of SEQ ID NO: 4 for optimal alignment. In another embodiment, the invention relates to an isolated polypeptide having DNA polymerase activity comprising a N-terminal 3′-5′ exonuclease domain having a exonuclease active site sequence motif L-G-V-D-L-E-T-T-G-L-D-P-H in a region of the polypeptide wherein the left-end residues L-G-V-D align with residues 29-32 of SEQ ID NO: 4, when the sequence of said polypeptide is aligned with the sequence of SEQ ID NO: 4 for optimal alignment.

The polymerase of the invention is in some embodiments suitably obtainable from certain Thermus species, such as, e.g., Thermus antranikianii, Thermus brockianus and closely related Thermus species. However, in useful embodiments the polymerase of the invention may be obtained directly from environmental samples, e.g. with methods such as described in WO 02/059351 which is incorporated herein by reference. Such environmental DNA samples may comprise DNA material from one or several species and they may originate from one or more Thermus species. The polymerases of the invention may be obtained from unclassified bacterial species. This includes bacterial strains belonging to the genus Thermus, such as shown by sequencing of the 16S rRNA gene, although said strains may not be identical to any of the previously characterized Thermus species.

Preferred polymerases of the invention have substantially higher activity than prior art polymerases. Preferably the polymerase of the invention has a specific activity of at least 1.000 U/mg when assayed as described in detail in Example 16 and more preferably at least 10.000 U/mg and even more preferably at least 15.000 U/mg such as at least 25.000 U/mg and more preferably at least 50.000 U/mg and yet more preferably at least 75.000 Units/mg such as at least 100.000 Units/mg. Preferred polymerases of the invention have at least 200.000 Units/mg when assayed as described herein, such as in the range of about 200.000-500.000 Units/mg.

In one embodiment, the polymerase of the present invention has a molecular weight of about 63 kDa as measured by SDS-PAGE gel electrophoresis and an inferred molecular weight from the nucleotide sequence of the gene.

The isolated polypeptides provided by the invention preferably have a pH optimum around pH 8.5 and a temperature optimum in the range of about 50-55° C.

In one aspect, the present invention relates to polypeptides having DNA polymerase strand displacement activity with a temperature optimum of at least 40° C., preferably the temperature optimum is in the range 50° C. to 70° C., more preferably in the range 50° C. to 60° C.

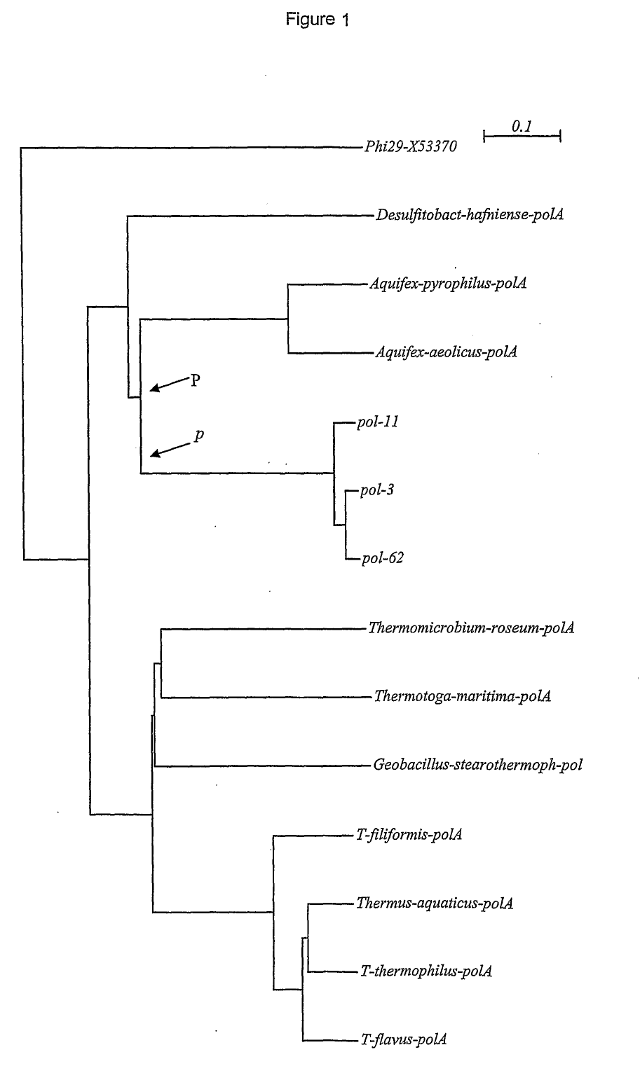

A conventional method of analysing evolutionary relationships of proteins and to characterize protein families is through the construction of phylogenetic trees. As seen in FIG. 1 the DNA polymerases of the invention form a distinct branch in a phylogenetic tree containing a wide selection of DNA polymerases including the closest known relatives from the bacterial species of the genus Aquifex and Desulfitobacterium hafniense. The three exemplified members of the DNA polymerases of the present invention appear as the so far only known representatives in this novel family and future member of this family can be indentified using the same method of constructing a phylogenetic tree. Thus the invention relates to isolated polypeptides having DNA polymerase activity and a polypeptide sequence such that when said sequence is included in alignment, together with the sequences of FIG. 1 using the alignment algorithm in the program ClustalX, and with a subsequent construction of phylogenetic tree, using the Neighbour Joining method, the said sequence will belong to the same branch as the sequences of the present invention. More specifically, the invention provides a novel sub-family sequence-based phylogenetic branch which is defined by a phylogenetic tree being prepared as described above, wherein said branch corresponds to internal branch p stemming from node P in the phylogenetic tree shown in FIG. 1

For construction of a phylogenetic tree, as in FIG. 1, the sequences are first aligned using the ClustalW algorithm (Thompson, J. D. et al., 1994) Higgins, D. G. and Gibson, T. J. (1994)) as implemented in the program ClustalX (Thompson, J. D., et al. (1997)) with default parameters. The aligned sequences are then used to create the phylogenetic tree with the neighbour joining method (Saitou, N, & Nei, M. (1987)) using the “Draw N-J Tree” option in ClustalX.

The polypeptides of the invention can be partially or substantially purified (e.g., purified to homogeneity), and/or are substantially free of other polypeptides. According to the invention, the amino acid sequence of the polypeptide can be that of the naturally occurring polypeptide or can comprise alterations therein. Polypeptides comprising alterations are referred to herein as “derivatives” of the native polypeptide. Such alterations include conservative or non-conservative amino acid substitutions, additions and deletions of one or more amino acids; however, such alterations should preserve the DNA polymerase strand displacement activity of the polypeptide, i.e., the altered or mutant polypeptides of the invention are active derivatives of the naturally occurring polypeptide having DNA polymerase strand displacement activity. Preferably, the amino acid substitutions are of minor nature, i.e. conservative amino acid substitutions that do not significantly alter the folding or activity of the polypeptide. Deletions are preferably small deletions, typically of one to 30 amino acids. Additions are preferably small amino- or carboxy-terminal extensions, such as amino-terminal methionine residue; a small linker peptide of up to about 25 residues; or a small extension that facilitates purification by changing net charge or another function, such as a poly-histidine tail, an antigenic epitope or a binding domain. The alteration(s) preferably preserve the three dimensional configuration of the active site of the native polypeptide, or can preferably preserve the activity of the polypeptide (e.g. any mutations preferably preserve the ability of the polypeptides of the present invention to catalyze DNA synthesis. The presence or absence of activity or activities of the polypeptide can be determined by various standard functional assays including, but not limited to, assays for binding activity or enzymatic activity.

Polypeptides of the invention may be modified to change their properties. This includes deletions, insertions and site-directed point mutations at one or more positions. An example of modification of this kind would be mutations of residues critical for 3′-exonuclease activity such as the residues functioning as ligands to the metal ions. An example of modification of Pol-11 of this kind would be one or more of the mutations Asp32 to Ala, Glu34 to Ala and Asp89 to Ala. Such modification is expected to decrease or abolish 3′-exonuclease activity and consequently reduce proofreading during template-directed DNA synthesis. However, a specific modification of this kind may in turn have an effect on the strand displacement properties, such as increasing processivity, which may be beneficial for certain applications.

Additionally included in the invention are active fragments of the polypeptides described herein, as well as fragments of the active derivatives described above. An “active fragment”, as referred to herein, is a portion of polypeptide (or a portion of an active derivative) that retains the polypeptide's DNA polymerase strand displacement activity, as described above. Appropriate amino acid alterations can be made on the basis of several criteria, including hydrophobicity, basic or acidic character, charge, polarity, size of side chain, the presence or absence of a functional group (e.g., —SH or a glycosylation site), and aromatic character. Assignment of various amino acids to similar groups based on the properties above will be readily apparent to the skilled artisan; further appropriate amino acid changes can also be found in Bowie, et al. 1990. For example, conservative amino acid replacements can be those that take place within a family of amino acids that are related in their side chains. Genetically encoded amino acids are generally divided into four families: (1) acidic=aspartate, glutamate; (2) basic=lysine, arginine, histidine; (3) nonpolar=alanine, valine, leucine, isoleucine, proline, phenylalanine, methionine, tryptophan; and (4) uncharged polar=glycine, asparagine, glutamine, cystine, serine, threonine, tyrosine. Phenylalanine, tryptophan and tyrosine are sometimes classified jointly as aromatic amino acids. For example, it is reasonable to expect that an isolated replacement of a leucine with an isoleucine or valine, an aspartate with a glutamate, a threonine with a serine or a similar conservative replacement of an amino acid with a structurally related amino acid will not have a major effect on activity or functionality. Consequently, the invention encompasses polypeptides with the sequences shown as SEQ ID NO: 4, SEQ ID NO: 5 and SEQ ID NO: 6 and substantially similar polymerase strand displacing active sequences having one or more conservative substitutions.

In one embodiment the polypeptides of the invention are fusion polypeptides comprising all or a portion (e.g., an active fragment) of an amino acid sequence of the invention fused to an additional component, with optional linker sequences. Additional components, such as radioisotopes and antigenic tags, can be selected to assist in the isolation or purification of the polypeptide or to extend the half-life of the polypeptide; for example, a hexahistidine tag would permit ready purification by nickel chromatography. The fusion protein can contain, e.g., a glutathione-S-transferase (GST), thioredoxin (TRX) or maltose binding protein (MBP) component to facilitate purification; kits for expression and purification of such fusion proteins are commercially available. The polypeptides of the invention can also be tagged with an epitope and subsequently purified using antibody specific to the epitope using art recognized methods. Additionally, all or a portion of the polypeptide can be fused to carrier molecules, such as immunoglobulins, for many purposes, including increasing the valency of protein binding sites. For example, the polypeptide or a portion thereof can be linked to the Fc portion of an immunoglobulin; for example, such a fusion could be to the Fc portion of an IgG molecule to create a bivalent form of the protein.

Also included in the invention are polypeptides having DNA polymerase strand displacement activity which have at least about 30% sequence identity (i.e., polypeptides which have substantial sequence identity) to the amino acid sequence of SEQ ID NO: 4 or SEQ ID NO: 5 described herein but preferably higher sequence identity, such as at least about 40% and more preferably at least about 50% or about 60% sequence identity and more preferably at least about 70% or about 75% sequence identity, and even more preferably at least about 80% or at least 90% sequence identity such as at least about 95% or 97% sequence identity such as at least about 99% sequence identity to said sequences. However, polypeptides exhibiting lower levels of overall sequence identity are also useful, particular if they exhibit higher identity over one or more particular domains or sequence motifs of the polypeptide, e.g. one or more of the motifs illustrated in FIG. 22. For example, polypeptides sharing high degrees of identity (e.g. over 80% or over 90%) over domains or sequence motifs necessary for particular activities, such as binding or enzymatic activity, are included herein.

Algorithms for sequence comparisons and calculation of “sequence identity” are known in the art as discussed above, such as BLAST, described in Altschul et al. 1990, or the Needleman and Wunsch algorithm (Needleman and Wunsch 1970) Generally, the default settings with respect to e.g. “scoring matrix” and “gap penalty” will be used for alignment. The percentage sequence identity values referred to herein refer to values as calculated with the Needleman and Wunsch algorithm such as implemented in the program Needle (Rice et al. 2000) using the default scoring matrix EBLOSUM62 for protein sequences, (or scoring matrix EDNAFULL for nucleotide sequences) with opening gap penalty set to 10.0 and gap extension penalty set to 0.5. The sequence identity is thus the percentage of identical matches between the two sequences over the aligned region including any gaps in the length. Percentage identity between two sequences in an alignment can also be counted by hand such as the sequence identity in an alignment that has been manually adjusted after automatic alignment.

Polypeptides described herein can be isolated from naturally-occurring sources (e.g., isolated from a bacterial species, such as in particular a thermophilic bacterium). Alternatively, the polypeptides can be chemically synthesized or recombinantly produced using the nucleic acids sequences of the present invention. For example, PCR primers can be designed to amplify an ORF from the start codon to stop codon, e.g. using DNA of a suitable source organism or respective recombinant clones as a template. The primers can contain suitable restriction sites for an efficient cloning into a suitable expression vector. The PCR product can be digested with the appropriate restriction enzyme and ligated between the corresponding restriction sites in the vector (the same restriction sites, or restriction sites producing the same cohesive ends or blunt end restriction sites).

Polypeptides described herein may be produced from any of a variety of microorganisms, either microorganisms that naturally contain in their genome nucleic acid sequences encoding the polypeptides of the invention or microorganisms into which a nucleic acid has been inserted, which encodes a polypeptide of the invention.

A polypeptide of the present invention may be a bacterial polypeptide. For example, the bacterial source may be a gram positive bacteria such as Bacillus, e.g. Bacillus stearothermophilus, Bacillus megaterium or Bacillus thuringiensis; or Streptomyces, e.g. Streptomyces lividans; or a gram negative bacterium such as E. coli, Pseudomonas sp.; Thermus, e.g. Thermus aquaticus, Thermus thermophilus or Thermus scotoductus or a Rhodothermus species; e.g. Rhodothermus marinus.

It is further contemplated that polypeptides of the present invention may be obtained from an Archaea such as a Sulfolobus species, e.g. Sulfolobus acidocaldarius or Sulfolobus solfataricus; a Pyrobaculum species, e.g. Pyobaculum islandicum or Pyrobaculum aerophilum; a Methanococcus species or a Halobacterium species.

A polypeptide of the present invention may be obtained from a microorganisms isolated from nature, e.g. from water or soil, including unclassified microorganisms or uncultivable or previously uncultured microorganisms, such as from environmental samples.

A polypeptide of the present invention may be encoded by a gene in an extrachromosomal genetic element such as a plasmid, including plasmids found in bacteria such as Thermus species.

A polypeptide of the present invention may be obtained from a non-bacterial source including eukaryotic organisms such as Fungi, including yeast, plants and animals.

A polypeptide of the present invention may be a viral polypeptide. For example, the viral source may be a bacteriophage having a bacterial such as E. coli or a thermophilic bacteriophage having a thermophilic bacterial host such as a Thermus species or Bacillus species. The viral source may also be a virus having a Eukaryotic host.

A polypeptide of the present invention may be obtained using nucleic acid probes designed to identify and clone DNA encoding polypeptides having DNA polymerase strand displacement activity using methods known in the art. A polypeptide of the invention can thus be obtained from different genera or species, including from DNA isolated directly from environmental samples or DNA identified from screening genomic or cDNA libraries. In a preferred embodiment, a nucleic acid probe is a nucleic acid sequence of the present invention or a portion thereof such as any of the sequences SEQ ID NO: 1 or SEQ ID NO: 2 or SEQ ID NO: 3, or a portion thereof, or a nucleic acid which encodes the polypeptide of the invention such as any of the polypeptides shown as SEQ ID NO: 4 or SEQ ID NO: 5 or SEQ ID NO: 6, or a subsequence thereof.

The polypeptides of the present invention can be isolated or purified (e.g., to homogeneity) from cell culture (e.g., from culture of bacteria) by a variety of processes. These include, but are not limited to, anion or cation exchange chromatography, ethanol precipitation, affinity chromatography and high performance liquid chromatography (HPLC). The particular method used will depend upon the properties of the polypeptide; appropriate methods will be readily apparent to those skilled in the art. For example, with respect to protein or polypeptide identification, bands identified by gel analysis can be isolated and purified by HPLC, and the resulting purified protein can be sequenced. Alternatively, the purified protein can be enzymatically digested by methods known in the art to produce polypeptide fragments which can be sequenced. The sequencing can be performed, for example, by the methods of Wilm, et al. (Nature, 379:466-469 (1996)). The protein can be isolated by conventional means of protein biochemistry and purification to obtain a substantially pure product, i.e., 80, 95 or 99% free of cell component contaminants, as described in Jacoby, Methods in Enzymology, Volume 104, Academic Press, New York (1984); Scopes, Protein Purification, Principles and Practice, 2nd Edition, Springer-Verlag, New York (1987); and Deutscher (ed.), Guide to Protein Purification, Methods in Enzymology, Vol. 182 (1990).

### Applications Using the Polypeptides of the Invention

The special properties of the isolated polypeptides of the invention as compared to counterparts in the prior art are beneficial for use in various methods. It is an object of the invention to provide methods using the isolated polypeptides of the invention. The methods include methods wherein the isolated polypeptides are used to catalyze DNA synthesis by addition of deoxynucleotides to the 3′ end of a polynucleotide chain, using a complementary polynucleotide strands as a template. In one embodiment of the invention, genetic material, such as genomic DNA, is amplified using the disclosed isolated polypeptides in a reaction wherein the genetic material is amplified through strand displacement reaction. Methods of this type using other less proficient DNA polymerases are well known in the prior art, for example “Rolling Circle Amplification” on circular DNA templates (Nelson et al. 2002; Dean et al. 2001; Alsmadi et al. 2003, Detter et al. 2002), “Hyperbranched Strand Displacement Amplification” (Lage et al. 2003) “Multiple Displacement Amplification” (Dean et al. 2002; U.S. Pat. Nos. 6,617,137 and 6,124,120) and “Strand displacement amplification” (see Walker et al., 1992 and U.S. Pat. No. 5,270,184).

The methods provided by invention include for example a method of amplifying a target nucleic acid sequence, the method comprising: bringing into contact a set of primers, DNA polymerase, and a target sample, and incubating the target sample under conditions that promote replication of the target sequence, wherein replication of the target sequence results in replicated strands, wherein during replication at least one of the replicated strands is displaced from the target sequence by strand displacement replication of another replicated strand, and wherein the DNA polymerase is an isolated polypeptide of the present invention. In preferred embodiments of the methods is the amplification performed at higher temperatures, such as above 50° C. up to about 95° C., preferably in the temperature range 50 to 70° C. The methods may also involve the use of various temperatures in different steps, including thermocycling.

The methods provide may involve amplification with and without primers. For example amplification of activated DNA can be done without addition of primers and rolling-circle of plasmids can be done without primers using plasmid DNA that has been nicked, for example relaxed plasmid DNA containing one or more nicks, i.e. break of one or the other strand of the nucleic acid.

The methods of the invention may involve amplification proceeding without strand displacement such as using single-stranded template. The methods provided may also involve different phases of amplification with and without strand displacement. For example, amplification of activated DNA without primers may start with an initial phase of DNA synthesis through filling of gaps in the DNA template, without strand displacement, and then a second and slower phase involves strand displacement in amplification of the initial DNA template. The third phase may then involve further strand displacement amplification after re-priming through hybridization of previously displaced strands.

The methods of the invention may involve the combined use of other polypeptides together with the polypeptides of the invention. A non-limiting example of this is the use of DNA helicases and/or single-strand DNA binding proteins to facilitate strand displacement.

The methods provided can be used for amplification of genetic material such as genomic DNA such as human genomic DNA. The DNA material which is being amplified can be from various sources, for example environmental samples, clinical samples, forensic samples and DNA samples isolated from organisms grown in a laboratory. The DNA can be relatively inaccessible in these samples. The methods provided may involve various treatments of samples in order to for example make the DNA more accessible for amplification. The use of the DNA polymerases of the invention may make possible the use of wider range of conditions not suitable for prior art strand displacing DNA polymerase such as Phi29 DNA polymerase. Higher temperatures can be useful in the treatment of the samples for example due to increased solubility of sample components, reduced viscosity and reduced risk of microbial contamination. Thermostable proteins are also generally more resistant to harsh conditions other than high temperatures such as conditions with relatively high concentrations of organic solvents (Bruins et al. 2001; Vieille and Zeikus 2001). The use of the polypeptides of the invention may also allow combined treatment of the sample, for example to solubilize the DNA, and simultaneous amplification and thus reduce the steps involved in the procedure which can for example simplify diagnostic applications, such as in clinical settings.

The methods provided can be used to amplify genetic material in samples containing limited amount of DNA such as amounts too limited for subsequent analysis or other manipulation. The analysis of the amplified genetic material includes genotyping techniques such as determination and screening for single nucleotide polymorphism.

The methods provided make use of the polypeptides of the invention and may involve the use of various other compounds including nucleic acid templates, oligonucleotide primers, labeled or unlabeled nucleotides, and stabilizing compounds such as polyethanol glycol, glycerol, amino acids or proteins such as bovine serum albumin. The nucleic acid may be modified in different was for various purposes. Nucleic acid primers can for example be modified to be resistant to exonuclease activity or to change specificity of priming.

A polypeptide of the invention can be used in applications with other enzymes and polypeptides. Examples of enzymes that can be used with a polymerase of the invention are DNA Helicases, single-strand DNA binding proteins, RNA ligases, DNA ligases, restriction enzymes, exonucleases, DNA polymerases, RNA polymerases and phosphatases. A polypeptide provided by the invention can suitably be used in combination with such enzymes as well as other components in kits for various applications.

Also provided are kits for use in practicing the methods of the subject invention. The subject kits typically include at least an isolated polypeptide DNA polymerase strand displacement activity, as described above, and a suitable reaction buffer. The kit may also include nucleotides or nucleotide analogs, e.g. nucleotide triphosphates such as dATP, dCTP, dTTP and dGTP, labeled or unlabeled. The subject kits may further include additional reagents necessary and/or desirable for use in practicing the subject methods, where additional reagents of interest include: an aqueous buffer medium (either prepared or present in its constituent components, where one or more of the components may be premixed or all of the components may be separate); RNase inhibitors, control substrates, control nucleic acids, template nucleic acids, primer oligonucleotides, and the like. The subject kits may also include other polypeptides having various other enzymatic activities. These activities include, but are not limited to, helicase activity, DNA binding activity, ligase activity, polymerase activity and nuclease activity and other activities of polypeptides having enzymatic activity on nucleic acids. Examples of enzymes having those activities are DNA helicases, single-strand DNA binding proteins, restriction enzymes, RNA ligases, DNA ligases, endonucleases, exonucleases, DNA polymerases, RNA polymerases and phosphatases. The various reagent components of the kits may be present in separated containers, or may all be pre-combined into a reagent mixture for combination with to be labeled ribonucleic acid. A set of instructions will also typically be included, where the instructions may be associated with a package insert and/or the packaging of the kit or the components thereof.

The references cited herein are incorporated by reference in their entirety. While this invention has been particularly shown and described with references to preferred embodiments thereof, it will be understood by those skilled in the art that various changes in form and details may be made therein without departing from the spirit and scope of the invention as defined by the appended claims.

The following Examples are offered for the purpose of illustrating the present invention and are not to be construed to limit the scope of this invention.

### EXAMPLES

### Example 1

### Bacterial Strains and DNA Isolation

In this work a number of strains of Thermus and Meiothermus were used. The strains were from the collection of Prokaria ltd. and represented all described Thermus species as well as few Meiothermus spp. The strains were all isolated from various geothermal fields in Iceland except for some of the reference strains. The selection of strains was based on the genetic relationship of 101 Icelandic Thermus strains based on a MEE analysis of 10 enzyme loci reported by Skirnisdottir et al. 2001 (Skirnisdottir, 2001) as well as on the DNA polymerase activity screening of thermophilic DNA polymerases from Icelandic Thermus strains reported by Hjorleifsdottir et al. 1997 (Hjorleifsdottir et al., 1997). Type strains of the following Thermus species were used as reference in the study: Thermus aquaticus strain YT-1 (DSM 625; type strain), Thermus brockianus strain YS38 (NCIMB 12676; type strain), Thermus filiformis strain Wal33 A.1 (DSM 4687, type strain), Thermus thermophilus strain HB8 (ATCC 27634, DSM 579; type strain), Thermus antranikianii strain HN3-7 (DSM 12462; type strain), Thermus scotoductus strain SE-1 (ATCC 51532; type strain), T. igniterrae strain 165 from the Prokaria strain collection was used instead of the type strain but it has 99% sequence identity to it (based on 16S rRNA gene sequence). Similarly, strain 51 from the Prokaria strain collection was used as it has 99% identity to the T. oshimai type strain.

The strains used in the present study were isolated at different temperatures (65° C., 72° C. and 80° C.). The strains were purified by repeated streaking onto medium 160 and 166 (Degryse et al., 1978; Hjorleifsdottir et al., 2001). DNA was isolated from the cultivated strains with Dynabeads DNA Direct kit according to the manufacturer's instructions (Dynal). DNA was also isolated from complex biomass samples. The hot springs used for collection of complex biomass samples were of various temperatures between 80-10° C. and pH, 2.1-8.5. DNA isolation from these samples was according to Marteinsson et al. (Marteinsson et al., 2001).

### Example 2

### Amplification of Gene Fragments and Construction of Gene Libraries

DNA polymerases of family A (Braithwaite and Ito, 1993) have shown to contain 3 conserved sites in the active site of the polymerase domain of the gene (Joyce and Steitz, 1994). The conserved motifs in the active site were used to design degenerate CODEHOP primers (Rose et al., 1998) flanking the region between motifs A and C. The primers used gave approximately 600 base long sequences: A-forw. 5′-GCCGCCGACTACTCCcarathgarht-3′ and C-rev. 5′-cangtrctrctCTACCACAAGCTCCCG-3′. DyNAzyme™ DNA polymerase (Finnzymes) was used as described by the manufacturer. The PCR reaction was done as follows: 94° C. for 5 min, before 30 circles of 94° C. for 50 s, 50° C. for 1 min, 72° C. for 1.5 min and at the end of the program an elongation step at 72° C. for 7 min. In cases when 600 base long PCR products were not retrieved, the annealing temperatures were varied by using a gradient from 40° C. up to 60° C. PCR products were separated on 1% TAE gels and bands of approximately 600 bases excised from the gel and purified by using GFX, PCR DNA and Gel Band Purification kit (Amersham-Pharmacia) according the manufacturer. Purified PCR products were cloned by using TOPO-TA Cloning Kit (Invitrogen) according the manufacturer. Cycle sequencing reaction was performed by using BigDye Terminator Cycle Sequencing Ready Reaction kit according to the manufacturer (PE Applied Biosystems) using the M13 forward and reverse primers.

Amplification of the gene fragments of DNA polymerase was successful from most of the strains but only from few of the environmental samples.

### Example 3

### Diversity Analysis of the DNA Polymerase Gene Fragments

Partial sequencing of the DNA polymerase gene was carried out on 2-8 clones and nucleotide sequences from each strain grouped by using 98% cutoff value in the Sequencer 3.1 software. The consensus sequence of each group was BLAST searched on amino acid level against NCBI Protein Database and closest sequences identified found and collected. The amino acid sequences were then aligned by ClustalX and phylogenetic tree created by using the Neighbor Joining Method. DNA polymerase sequences from representative strains were used for the creation of the polymerase tree. The GeneBank accession numbers of the polymerase sequences used had the following accession numbers Thermus aquaticus AAA27507, Thermus thermophilus P52028, Thermus flavus P30313, Thermus filiformis AAC46079, Aquifex aeoliticus NP214348.

Some strains revealed a novel type of DNA polymerase, which did not show close relation to Taq DNA polymerase I. Some of the strains gave both the expected Taq like polymerase and also the novel type of polymerase gene showing closest sequence identify of 30-35% to Aquifex DNA polymerase (BLAST alignment).

DNA polymerase fragments were only successfully amplified from few of the environmental samples but nevertheless the novel type of DNA polymerase was also found in these samples (polymerase Pol-62, SEQ ID NO: 3). The phylogenetic tree in FIG. 1 shows the phylogenetic relationship of the gene products of the invention to a number of public and prior art DNA polymerases sequences, including Aquifex DNA polymerase which is the closest known relative.

### Example 4

### Whole Gene Retrieval

The approximately 600 bp sequences retrieved from the sequencing of the polymerase gene library were used as templates for designing two sets of specific inverse primers. One set downstream of the 3′-end and another set upstream of the 5′ end of the sequence. In addition to the specific primers three arbitrary primers were used;

| | |
|---|---|
| Arb1: | 5′-GGCCACGCGTCGACTAGTACNNNNNNNNNNGATAT-3′, |
| Arb.2: | 5′-GGGCACGCGTCGACTAGTACNNNNNNNNNNACGCC-3, |
| Arb.3: | 5′-GGCCACGCGTCGACTAGTAC-3′. |

The GENEMINING method is a gene walking method consisting of two PCR reactions were one gene specific primer and one arbitrary primer is used in each reaction creating flanking sequences. Two rounds of PCR amplifications were used according to the previously described arbitrary primer PCR method (Caetano-Anolles, 1996; Pratt and Kolter, 1998). After excising the PCR bands from 1% TAE gels they were purified by using GFX, PCR DNA and Gel Band Purification kit (Amersham-Pharmacia) according the manufacturer. The purified PCR products was cloned by using TOPO TA Cloning Kit (Invitrogen) according the manufacturer. Colonies were picked and cycle sequencing done by using M13 forward/rev primers and BigDye Terminator Cycle Sequencing Ready Reaction kit according to the manufacturer (PE Applied Biosystems). When sequences had been assembled with the first sequence in the Sequencer 3.1 software a new set of specific inverse primers were designed and the process repeated until the whole gene was retrieved.

### Example 5

### Hybridization

A new type of DNA polymerase genes was observed in some of the Thermus strains when using the degenerate primers from the conserved regions of the polymerase domain. For confirming the placement of the new type of polymerase gene in the genomes of the different Thermus species, a hybridization experiments were performed.

The hydridization experiment was started with PCR amplification. Specific primers were designed based on the sequences obtained with the degenerate primers. The primers were forward: 5′-acgccctcaccgccagcctggtcc-3 and reverse: 5′-ttctcccagaggagggccagggccat-3′ covering a 340 bp sequence. Two or three strains out of each of the Icelandic Thermus. spp. were used as well as the type strains of most species. The amplified PCR products were run on agarose gels. Nucleic acid bands were blotted onto nylon transfer membrane Hybond-N+ (RPN203B) from Amersham Pharmacia Biotech according to their protocol. The probe was made from nested PCR using the above primers on template, which was amplified from the retrieved new polymerase ORF of one strain. The probe was labeled with DIG-High Prime kit from Roche. Hybridization was according to DIG detection kit protocol (Roche). Those strains which showed a hybridization signal indicating that the PCR product was the new polymerase gene were concluded positive.

The results of PCR and hybridization are indicated in Table 2.

TABLE 2
PCR amplification of a new type of polymerase
fragment and hybridization

| | | PCR | Hybridization |
|---|---|---|---|
| Strain | Species | product | signal |
| 2120 | Thermus antranikianii | + | + |
| 2945 | Thermus antranikianii | + | − |
| 74 | Thermus aquaticus | − | − |
| 253 | Thermus brockianus | (+) | + |
| 79 | Thermus brockianus | − | − |
| 133 | Thermus brockianus | + | + |
| 140 | Thermus brockianus | + | + |
| 284 | “Thermus eggertsonii”* | − | − |
| 2789 | Thermus eggertsonii* | − | − |
| 947 | Thermus filiformis | − | − |
| 1087 | Thermus flavus | − | − |
| 165 | Thermus igniterrae | − | − |
| 3040 | Thermus igniterrae | − | − |
| 73 | Thermus oshimai | + | + |
| 219 | Thermus oshimai | − | − |
| 52 | Thermus scotoductus | + | + |
| 53 | Thermus scotoductus | − | − |
| 346 | Thermus scotoductus | − | − |
| 72 | Thermus thermophilus | − | − |
| 945 | Thermus thermophilus | − | − |

*Thermus eggertssonii is a potentially new species described at Prokaria but has not been published.

As indicated in Table 2 T. aquaticus, T. thermophilus, T. filiformis, T. eggertssonii, T. filiformis and T. igniterrae are all negative in containing the new type of DNA polymerase gene. The other four species T. scotoductus, T. brockianus, T. oshimai and T. antranikianii all have both strains with and without the new polymerase gene.

### Example 6

### Cloning of DNA Polymerase Genes, Expression and Activity Measurements

After retrieving the whole genes of the novel type of DNA polymerases from strains and biomass the genes were cloned into expression vectors for producing the enzymes. The respective polymerase gene was cloned into the expression vectors pBTac1 (Amann et al., 1983) and pJOE3075 (Wilms et al., 2001) with and without histidine tail fusion. E. coli BL21 cells were transformed with the corresponding vector constructs. For confirming expression of the cloned DNA polymerase cells were cultivated and crude extracts run on SDS gels. In case an increased band was observed on SDS gel compared to negative control sample (same cells without expression vector) the crude extract was heated at 60° C. for 15 minutes to inactivate the E. coli DNA polymerase and then the polymerase activity was tested. The standard DNA polymerase assay was incorporation of 3H-labeled nucleotides into partially digested calf thymus DNA as described previously by Hjorleifsdottir et al. (Hjorleifsdottir et al., 1997). The reaction mixture (60 μl) was incubated at 60-65° C. for 15 min.

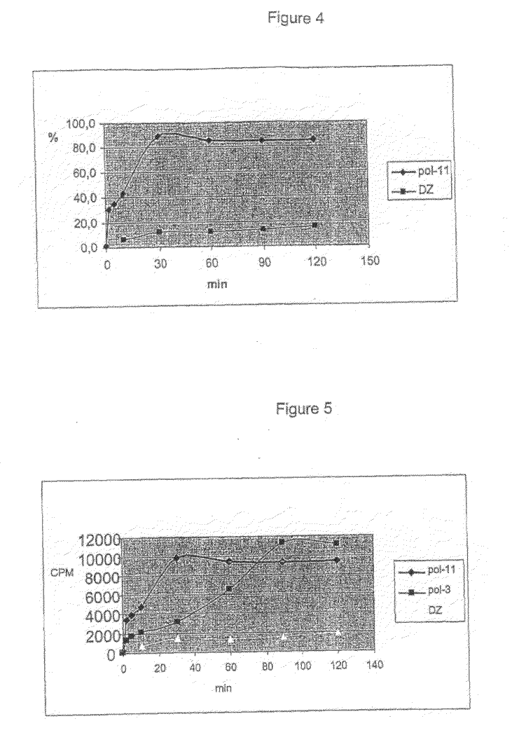

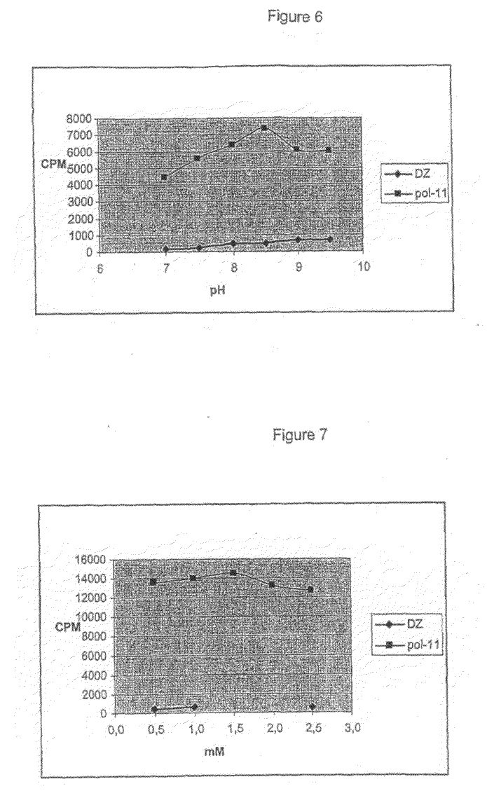

Activity of Pol-11 is shown in FIG. 2. Pol-11 showed a very steep increase of nucleotide incorporation during the first two minutes but not as steep and linear increase during the rest of the time. Time for the activity assay varied between samples. It was usually enough to have 10 min at 55° C. to reach a plateau as seen in FIG. 4. However, after the enzymes had been purified (on a histidine affinity column) and compared again the steep increase is still the first two minutes but increased incorporation was observed for the first 30 min. for Pol-11 but Pol-3 continued to incorporate nucleotides as long as any were left as can be seen in FIG. 5.

### Example 7

### Initial Characterization of Enzyme Properties

Optimal reaction temperature was found by incubating the enzymes in 40°, 45°, 50°, 55°, 60°, 65°, 70° and 75° C. The temperature optimum of Pol-11 was 50-55° C.° C. as shown in FIG. 3.

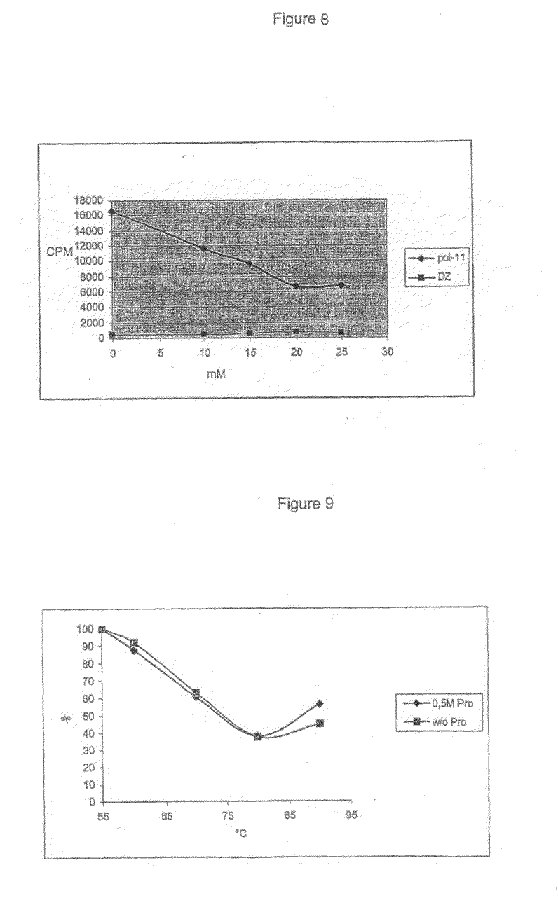

Optimal pH of the buffer was tested by using reaction conditions of 10 mM Tris-HCl buffer, 1.5 mM MgCl₂, 50 mM KCl and 0.1% Triton-X-100 which was adjusted to pH 7.0-9.5 with 0.5 point intervals increase. Optimal pH was 8.5 for Pol-11 in Tris-HCl buffer (FIG. 8). Two other buffers were tested (MOPS and Glycine buffer) It seemed that glycine buffer was better than Tris-HCl and it gave also optimal pH at 8.5.

Optimal MgCl₂ concentration of the buffer was tested by increasing the MgCl₂ concentration from 0.5 to 2.5 mM in final reaction buffer with 0.5 mM adjustments. Optimal MgCl₂ concentration was found to be 1.5 mM final concentration (FIG. 9) of the reaction mixture but very little difference was observed from 0.5-2.5 mM concentrations.

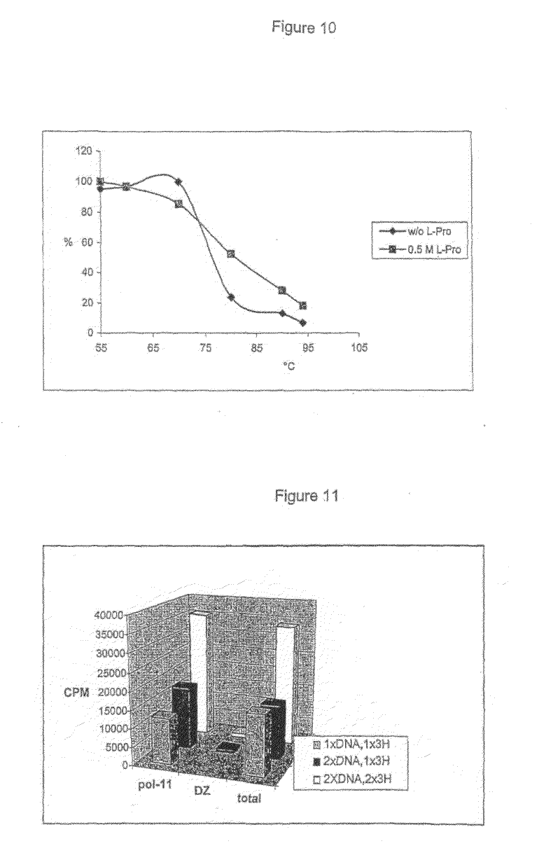

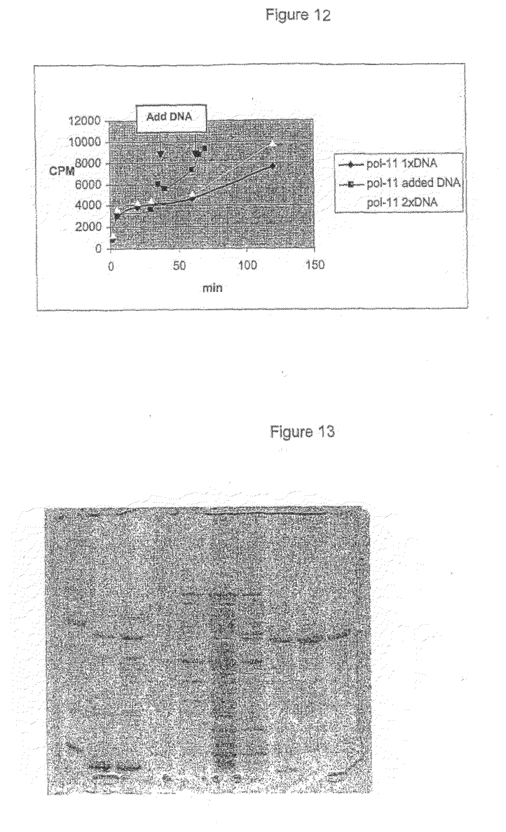

Optimal ammonium sulfate ((NH₄)₂SO₄) concentration was tested by increasing the concentration of this salt from 0-2.5 mM with 0.5 mM adjustments. Optimal ((NH₄)₂SO₄) concentration for the activity reaction was found to be 0 mM for Pol-11 (FIG. 10). Other buffers tested were MOPS 50 mM, MgCl₂ 1.5 mM, KCl 10 mM, BSA 25 mM and glycine buffer 25 mM, MgCl₂ 1.5 mM, KCl 50 mM, BSA 25 mM. In both buffers pH was varied from 7.0 to 9.5.

### Example 8

### Heat Stability

Heat stability of Pol-11 was studied in two experiments. The potentially stabilizing effects of the presence of proline in high concentrations was also investigated. In a first experiment, reactions were done (in triplicate) using the following mix:

| | | |
|---|---|---|
| 10× buffer | 5 | uL |
| tritium dTTP | 1 | uL |
| dNTP | 5 | uL (final 1 mM) |
| Enzyme | 1.1 | ug |
| L-Proline | 0.5 | M (+/−) |
| Salmon sperm activated DNA | 5 | uL (final 0.1 mg/ml) |
| H₂O | to 50 | uL |

The reaction were performed at 55, 60, 70, 80 and 90° C. for 20 minutes, respectively, and then cooled on ice. 20 uL of the mixture were dispensed on DE81 ion exchange filters and washed twice in 100 mM phosphate buffer (pH 7).

The filters were dried and radioactivity counted in liquid scintillation counter. The relative activity with and without L-proline as also shown in FIG. 9 was as follows:

TABLE 3

| Temperature | with L-proline | without L-proline |
|---|---|---|
| 55° C. | 100 | 100 |
| 60° C. | 87.6 | 91.9 |
| 70° C. | 60.7 | 63.3 |
| 80° C. | 37.8 | 37.4 |
| 90° C. | 56.3 | 45.0 |

The results show that substantial activity was observed even at the highest temperatures. Under the conditions of this experiment, the effect of L-proline is marginal.

In a different experiment, Pol-11 was incubated at a given temperature for 15 minutes and then activity was determined at 55° C. for 20 minutes. The initial mixture was made as follows:

| dNTP | 5 | uM |
|---|---|---|
| Tritium dTTP | 2 | uM |
| Enzyme | 1 ((1.1 ug Pol-11) | |
| 10× Pol buffer | 5 | |
| L-Proline | 0.5M (with and without proline) | |
| ssDNA oligomer | 1 | uM |
| H₂O | to 40 | uL |

The samples were heated for 15 minutes at 55, 60, 70, 80, 90, 94° C. for 15 minutes and then 10 uL of activated Salmon sperm DNA was added (final conc 0.5 mg/ml) to determine activity by continued incubation at 55° C. for 20 minutes. The samples were then cooled on ice and 20 uL dispensed on DE81 ion exchange filters and wash twice in 100 mM phosphate buffer (pH 7). The filters were dried and radioactivity counted in liquid scintillation counter.

The relative activity as, also shown in FIG. 10, was as shown in Table 4.

TABLE 4

| Temperature: | Act. without L-proline | Act. with L-proline |
|---|---|---|
| 55° C. | 95.2 | 100 |
| 60° C. | 96.6 | 96.8 |
| 70° C. | 100 | 85.5 |
| 80° C. | 23.8 | 52.5 |
| 90° C. | 13.0 | 28.2 |
| 94° C. | 6.8 | 18.3 |

According to the results, Pol-11 does not loose all activity after incubation for 15 minutes up to 94° C. Residual activity is substantially higher after incubation in the presence of L-proline at temperatures above 80° C.

### Example 9

### Addition of Template DNA and Nucleotides

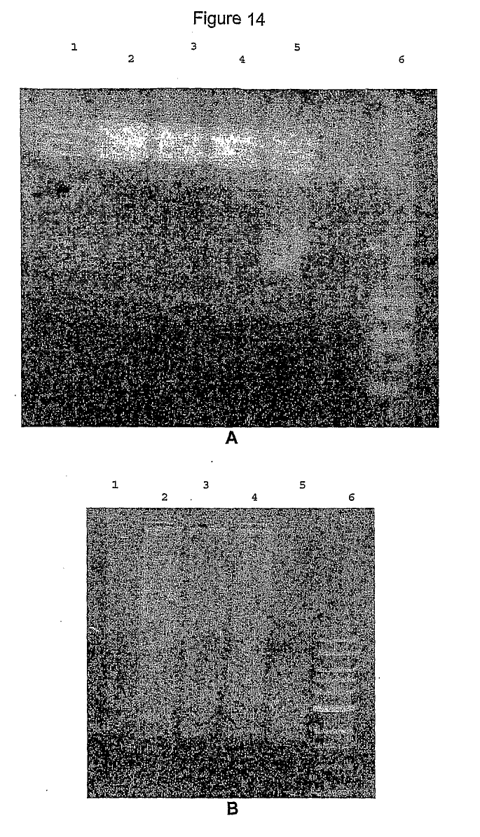

The effect of adding template DNA was tested by adding DNA to the reaction after 30 min and after 60 min. Also it was tested to use double amount of DNA template in the beginning of the reaction. Doubling the amount of labeled nucleotide was also done. Results showed (FIG. 13) that increasing template DNA increases the activity and doubling the labeled nucleotide doubles the incorporation. By adding template DNA to the reaction at different time intervals a sudden increase of incorporation was observed (FIG. 14). This is in agreement with what is usually observed in the activity assays where a very steep incorporation is observed during the first 2 minutes.

### Example 10

### Expression and Purification

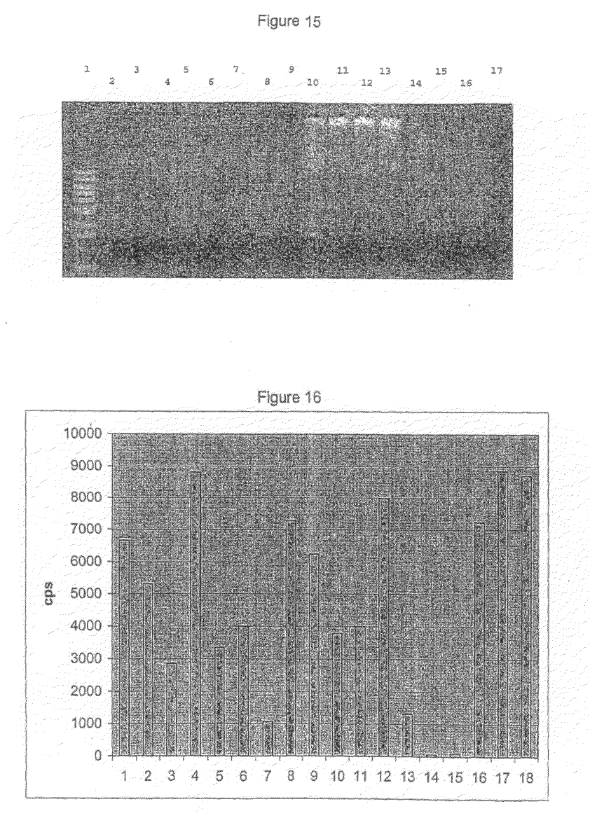

For further analyzes it was decided to purify polymerase Pol-11. A recombinant strain pAP18b of E. coli BL-21 RIL (Stratagene) with pJOE3075 with histidine tail fusion was used. The purification was done by heating at 65° C. for 10 min. and centrifuging precipitate. Effluent was run through histidine affinity column HiTrap Chelating HP (Amersham cat. no 17-0408-01). The running buffer was 20 mM NaPO₄, 500 mM NaCl, 10 mM Imidazol pH 7.6. The elution buffer was 20 mM NaPO₄, 500 mM NaCl, 500 mM Imidazol pH 7.6. In a step elution 20% was used for eluting loosely bound proteins and 40% elution buffer for final elution. The polymerase fractions were collected and dialyzed in storage buffer containing 40 mM Tris-HCl (pH 7.4) 0.2 mM EDTA, 200 mM KCl. The fractions were run on SDS gel for confirming purification of the enzyme. Before freezing the sample was mixed with glycerol (50% final concentration) with a final protein concentration of 0.2 mg/ml. Fractions from the HiTrap Chelatin HP column were run on SDS gel. As shown on FIG. 15 there is a substantially pure product in the final fractions which were pooled and used for all further experiments of Pol-11.

### Example 11

### Location of Novel Polymerase Genes

Early in the study the question arose if the gene of the new polymerase could be located on a plasmid since it was found in some Thermus species but not others. Specific experiments were made to clarify this question as outlined below in sections A to D.

A) chromosomal DNA was hybridized with labeled fragment of the new polymerase gene. Hybridization of total DNA gave bands which are above 12 Kb (data not shown).
B) DNA was isolated from 3 strains containing the new polymerase gene and 3 stains which do not contain it. The total DNA was run on 0.5% agarose gel over night at 25 V. A plasmid like band observed above 12 kb in one of the positive strains was cleaved out and DNA isolated using GFX, PCR DNA and Gel Band Purification kit (Amersham-Pharmacia) according the manufacturer. The DNA was used as template for PCR using ORF primers of the new plasmid. These were 140polAq-Nde-F 5′-cgaattccatatggaggggtttgaactccactac-3′ and 2120polAq-BglII-R 5′-cgcagatcttcatgcctcctcccacggcg-3′. In case some chromosomal DNA was mixed with the prospective plasmid DNA exonuclease III and exonuclease I were mixed with the GFX purified DNA to digest all linear DNA which might have been in the sample. PCR reaction with the same ORF primers was repeated.

Two of the 6 strains run on 0.5% agarose gel showed bands which could be plasmids. Strain 140, which contains the new polymerase, had a plasmid-like band above 12 KB and strain 72, which does not contain the new polymerase, had a band of 7 KB. The 12 KB band was cleaved out of the gel and PCR done on the purified DNA template. PCR products of approximately 1700 bp were observed, the same size as the positive control which was DNA from strain 140. The second PCR reaction done on the template after exonuclease treatment also gave a PCR product of the correct size.

C) Stretches of sequences on both sides of the ORF of the new polymerase genes were sequenced. These 450 bp and 510 bp sequences were compared to known bacterial sequences by BLAST. This was done to confirm if these sequences were of known Bacterial origin. The BLAST search of the 450 bp/510 bp on either side of the new gene did not give any known identity of bacterial or other origin.

### Example 12

### Incorporation of Tritium dTTP into Nicked (“Activated”) Calf Thymus DNA

Incorporation of tritium dTTP by DNA polymerase pol 11 and Thermus eggertssonii (Teg) DNA polymerase were tested with the following reaction:

| | |
|---|---|
| Calf thymus DNA: | 6 ul |
| dNTP (10 mM): | 6 ul |
| 10× Teg buffer: | 6 ul |
| H₂O: | 32 ul |
| Enzyme solution: | 10 ul |

The 10×Teg buffer (975 ul) was supplemented with tritium dTTP (25 ul)-Amersham (cat no. TRK424). The enzyme solution consisted of 1 ul of enzyme diluted into 9 ul of H₂O. Unit activity of Teg polymerase was 3 U/ul. Amount of Pol-11 polymerase was 1.14 ug/ul.

Reaction components for several reactions were mixed (except enzyme) and dispensed (50 ul) into 1.5 ml Eppendorf tubes. Reactions were preheated at 55° C. in a water bath. Enzyme solution was added to reaction(s) and incubated at 55° C. for 0-90 min. Three reactions were performed for each time point. Control reactions were without enzyme. Reactions were terminated by adding EDTA (to 50 mM) and placed on ice. 50 ul were drawn from each reaction and dispensed on DE81 filters (Whatman). The filters were dried at 75° for 10 min and washed twice in a 100 mM phosphate buffer (pH 7.5).

The filters were placed in scintillation vials containing 5 ml of scintillation fluid (Packard Ampligold) and measured in a scintillation counter.

### Example 13

### Strand Displacement of Pol-11 and Primer Requirement Using a Single Strand Template

Extension was tested using the following reactions and conditions:

| | |
|---|---|
| Template: pUC 19 (10 ng) | 5 ul |
| dNTP (10 mM) | 2 ul |
| 10× Teg buffer* | 2 ul |
| M13 F primer | 1 ul |
| M13 R primer | 1 ul |
| H₂O | 7 ul |
| Pol-11 enzyme | 1 ul |
| | (added after heating at 94° C. for 4 min) |

Reaction components were mixed except enzyme solution and heated to 94° C. for 4 min. Reaction performed at 55° C. overnight (14 hours). A similar reaction was performed using Phi29 instead of Pol-11. In the Phi29 reaction, a 10× buffer for Phi29 was used instead of 10×Teg buffer and the reaction was performed at 29° C. overnight (14 hours). The results are shown in FIG. 14. No amplification was visible in samples without the Pol-11 enzyme. Slight amplification is visible in sample where no primers are applied. In samples with template, primers and enzyme, large amounts of high molecular weight material is synthesized. This happens regardless the use of 1 or 2 primers respectively.

### Example 14

### Nucleotide Requirement of Pol-1

Extension was tested using the following reactions and conditions:

| | |
|---|---|
| Template: pUC 19 (10 ng) | 5 ul |
| dNTP, none or A, T, G, C | 2 ul |
| respectively (10 mM) | |
| 10× Teg buffer* | 2 ul |
| M13 F primer | 1 ul |
| M13 R primer | 1 ul |
| H₂O | 7 ul |
| Pol-11 enzyme | 1 ul |
| | (added after heating at 94° C. for 4 min) |

Reaction components were mixed save enzyme and heated to 94° C. for 4 minutes. Reaction performed at 55° C. overnight (14 hours). The results are shown in FIG. 15.

### Example 15

### Exonuclease Activity of Pol-11

Tritium incorporation by Pol-11.

| | |
|---|---|
| Calf thymus DNA: | 10 ul |
| dNTP (10 mM*): | 10 ul |
| 10× Teg buffer: | 10 ul |
| H₂O: | 65 ul |
| Tritium dTTP** | 2 ul |
| Pol-11 Enzyme: | 3 ul |

*2.5 mM of each nucleotide,
**Amersham (cat no. TRK424).

Reaction placed under mineral oil in an Eppendorf tube and incubated at 55° C. overnight. Phenol/chloroform extraction (2×) to destroy Pol-11 activity. Ethanol precipitation and wash (3×) in 70% ethanol (wash away unincorporated nucleotides). Dissolve DNA in 100 ul TE. Measure incorporated tritium in scintillation counter and calibrate cpm per ul TE.
Exonuclease activity test.

| | |
|---|---|
| Calf thymus DNA (tritiated) | 5 ul |
| Calf thymus DNA (cold) | 2 ul |
| 10× Teg buffer | 2 ul |
| H₂O | 1 ul |
| dNTP (10 mM) or H₂O: | 5 ul |
| Pol-11 enzyme or H₂O | 5 ul |

The enzyme was added last with 1 ul of enzyme diluted into 4 ul of H₂O. The amount of pol 11 polymerase was 1.14 ug/ul. Samples heated or NOT heated to 94° C. for 4 minutes prior to addition of enzyme. Samples incubated at 55° C. overnight. Samples (10 ul) were drawn from each reaction and dispensed on DE81 filters (Whatman). The filters were dried at 750 for 10′ and washed twice in a 100 mM phosphate buffer (pH 7.5). The filters were placed in scintillation vials containing 5 ml of scintillation fluid (Packard Ampligold) and measured in a scintillation counter. The measured incorporation is shown in FIG. 16. The trend is for the cps to decrease in samples containing Pol-11. The decrease diminishes less in samples containing dNTP compared to samples without dNTP. Decline in counts was time dependant. There is a definite trend between decrease in cps whether dNTP is present or not. If the diminishing cps is due to Exonuclease activity, this activity can be assigned to Pol-11.

### Example 16

### Specific Activity of Pol-11

Two experiments were done to measure specific activity of Pol-11 and compare its activity to Phi29 DNA polymerase.
Experiment A: Specific activity determination of Pol-11 DNA polymerase using salmon sperm activated DNA.

| | | |
|---|---|---|
| DNA | 0.1 or 0.6 | mg/ml final concentration |
| 10× TEG buffer | 5 | μL |
| Pol-11 | 0.06 | μg |
| dNTP | 1 | mM final conc |
| | (total 50 | nmol per reaction) |
| tritium labeled dTTP | 2 | |
| Water | to 50 | μL |

Timer intervals were 0/5/10/15/30/60/120 minutes in 0.6 mg/ml DNA and 0/5/10/15/30/60/120/180/240/420 and 720 minutes in 0.1 mg/ml
Incubated at 55° C. and samples heated at 95° C. for 10 minutes and put on ice and spotted on DE-81 filters and washed twice in 100 mM phosphate buffer (pH 7) and dried and counted in a liquid scintillation counter.
The percentage total CPM at different reaction times using two different concentrations of template DNA were as shown in Table 5.

TABLE 5

| Time (minutes) | DNA 0.1 mg/ml | DNA 0.6 mg/ml |
|---|---|---|
| 0 | 0 | 0 |
| 5 | 6.680233 | 73.51422 |
| 10 | 6.952217 | 86.40363 |
| 15 | 10.27961 | 88.34588 |
| 30 | 11.63171 | 94.16392 |
| 60 | 14.56483 | 99.58429 |
| 120 | 26.55363 | 103.4171 |
| 180 | 37.42516 | |
| 240 | 40.95312 | |
| 360 | 81.03745 | |
| 480 | 96.13157 | |
| 720 | 102.2629 | |

Total for 0.1 mg/ml = 51106; Background 571.5 CPM
Total for 0.6 mg/ml 80006; Background 910.5 CPM

The results are also shown in FIG. 20.
Experiment B: Comparison of the activities of Pol-11 and Phi29 DNA polymerases using activated DNA without primers.

| | | |
|---|---|---|
| Activated DNA | 0.6 | mg/ml final concentration |
| 10× TEG/Phi29 buffer | 5 | μL |
| Pol-11/Phi29 | 0.02 | μg (Pol-11) and |
| | 0.1 | μg (Phi29) |
| dNTP | 1 | mM final conc |
| | (total 50 | nmol per reaction) |
| tritium labeled dTTP | 2 | |
| Water | to 50 | μL |

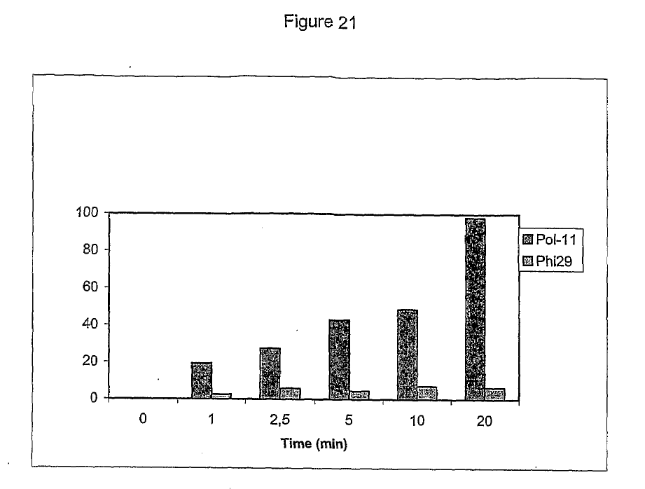

Incubated at 30° C. Phi29 and Pol-11 and spotted directly on DE-81 filters and washed twice in 100 mM phosphate buffer (pH 7) and dried and counted in a liquid scintillation counter. Time intervals were 0/1/2.5/5/10/20 minutes for samples. The result were as shown in Table 6 as also shown in FIG. 21:

TABLE 6

| Time (min) | Phi 29 | Pol-11 |
|---|---|---|
| 0 | 0 | 0 |
| 1 | 2.488893 | 19.2603 |
| 2.5 | 5.865876 | 27.52042 |
| 5 | 4.418598 | 42.93891 |
| 10 | 7.199643 | 48.90183 |
| 20 | 6.417828 | 98.15542 |

Total: 23492.37, background: 2788.333

The specific activity of Pol-11 in the two experiments corresponds to 360.000 units per mg protein, measured from the rate of incorporation during the first 10 minutes. The specific activity of Phi29 DNA polymerase is 10.800 units per mg.

### Example 17

### Amplification Using (Thiol) Hexamers

Template: human genomic DNA
Initial denaturation: 96° C., 4 minutes
Annealing time: 1 minute at 30° C.
Extension time: 5, 10 or 20 minutes at 55° C. respectively
Primers: hexamers, 10 pmol (total in each 20 ul reaction)
Cycles: 5, 10 or 20 respectively.
Reaction: A master mix was used to prevent errors. Each extension time was divided into 4×20 ul reactions. Each individual reaction consisted of:

| | | |
|---|---|---|
| Template: | 1 ul | (1 ng/ul or 5 ng/ul) |
| 10× Teg buffer | 2 ul | |
| dNTP (10 mM) | 2 ul | |
| Hexamers (10 uM) | 1 ul | |
| H₂O | 9 ul | |
| Total: | 15 ul | |

Denaturation step: 96° C. for 4 minutes (Ice)
Polymerase mixture addition:

| | |
|---|---|
| Polymerase Pol-11: | 0.5 ul |
| H₂O | 4.5 ul |
| Total: | 20 ul |

Incubation at 55° C. for 5, 10 or 20 cycles, respectively. Sample (20 ul) removal at each timepoint. Sample aliquots were frozen. Negative controls (reactions without Pol-11 enzyme) for each extension were incubated at reaction conditions for 20 cycles.

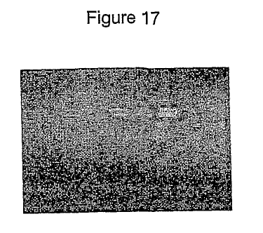

Increasing amounts of material synthesis could be observed on a time course basis. No amplification was observed in negative samples, see FIG. 17. Hexamers can be utilized to amplify human genomic DNA. Extension for 5 minutes for 5 cycles seems to generate quantities of material on par with longer (20 minute) extension cycles. This indicates that the Pol-11 enzyme works fast enough to allow shorter rather than longer extension times to generate long transcripts (in the form of high molecular weight DNA). Prolonged incubations with hexamers containing a thiol group backbone yield more high molecular weight material than identical incubations containing normal hexamers. The thiol based hexamers seem to be either more suitable for extension or less prone to exonuclease activity. It was noted when amplifying fragmented DNA with small oligos with low TM values we need extension time close to their optimum annealing temperatures to allow successful amplification (results not shown). Re-annealing and cycling is necessary to generate quantities of high molecular weight material from fragmented DNA. No such cycling is necessary in circular DNA. No high temperature template dissociation is required in successive cycling and re-annealing extensions, indicating that the generated high molecular weight material is to a large extent on single stranded form.

### Example 18

### Amplification of Beta-Actin Gene from Human Genomic DNA

The template used in this experiment was human genomic DNA amplified using Pol-11 as described in Example 17.

### Reaction:

| | | |
|---|---|---|
| Pol 11 amplified material: | 1 | ul |
| 10× Teg reaction buffer: | 2 | ul |
| B-actin F-primer (20 uM): | 1 | ul |
| Beta-actin R-primer (20 uM): | 1 | ul |
| dNTP (10 mM): | 0.3 | ul |
| Teg polymerase (3 U/ul): | 0.2 | ul |
| H₂O: | 9.5 | ul |

96° C., 4 min; 55° C. annealing temp.; 72° C. ext. temp, 1 min; 39 cycles.

The products were visualized by gel electrophoresis as shown in FIG. 18.

Bands of expected size appeared in samples with 5 ng of initial template. The PCR contained 1/20 of this amount or the equivalent of 0.4 ng of starting material in the Pol-11 amplified sample. The negative control samples were also subjected to the same PCR conditions with identical template dilution. This amount was not enough to generate a probable Beta-actin product by PCR. Untreated (original human genomic material) was successfully amplified by PCR. In a sample containing 0.125 ng of untreated material, expected bands appeared, albeit only barely visible.

To verify the amplification of the Beta-actin gene, Bands from samples amplified by Pol-11 and untreated samples were cloned and sequenced. In all clones Beta-actin sequence was confirmed. No abnormal mutations or mutation rates could be observed in bands originating from Pol-11 treated samples.

The residual template background from the original human genomic DNA was not enough to allow PCR amplification of probable Beta-actin band. A difference in terms of PCR products was observed between identical samples extended for different periods of time. A sample extended for 5 min during 20 extension cycles readily produced a Beta-actin band in a successive PCR with specific primers, whereas a identical sample extended for 10 min during 20 extension cycles rendered no Beta-actin band by PCR using the same master mix as the PCR from the less extended (5 min) Pol-11 template.
This indicates a difference in quality or quantity of suitable amplified material (the genomic region covering the Beta-actin gene being amplified) in terms of extension.

### Example 18

### Amplification of Human Genomic DNA and PCR Using Specific Primers

Human genome DNA was amplified with the following protocol

| | | |
|---|---|---|
| dNTP (each) | 250 | uM |
| 10× DNA pol b | 2 | uL |
| Pol-11 | 0.5 | uL |
| Template | 1-5 | ng |
| Random oktamers | 1-5 | pmol |
| H2O | to 20 | uL |
| Sample1 (no Pol-11) | 5 | ng DNA |
| Sample2 | 1 | ng DNA/1 pmol oktamers |
| Sample3 | 1 | ng DNA/5 pmol oktamers |
| Sample4 | 5 | ng DNA/1 pmol oktamers |
| Sample5 | 5 | ng DNA/5 pmol oktamers |

Heat at 94° C. for 5 min and allow to anneal to RT for 10 min and then add Pol-11.

### Thermal Program:

| | |
|---|---|
| 55° C. | 20 min |
| 20° C. | 2 min |
| 5 cycles | |

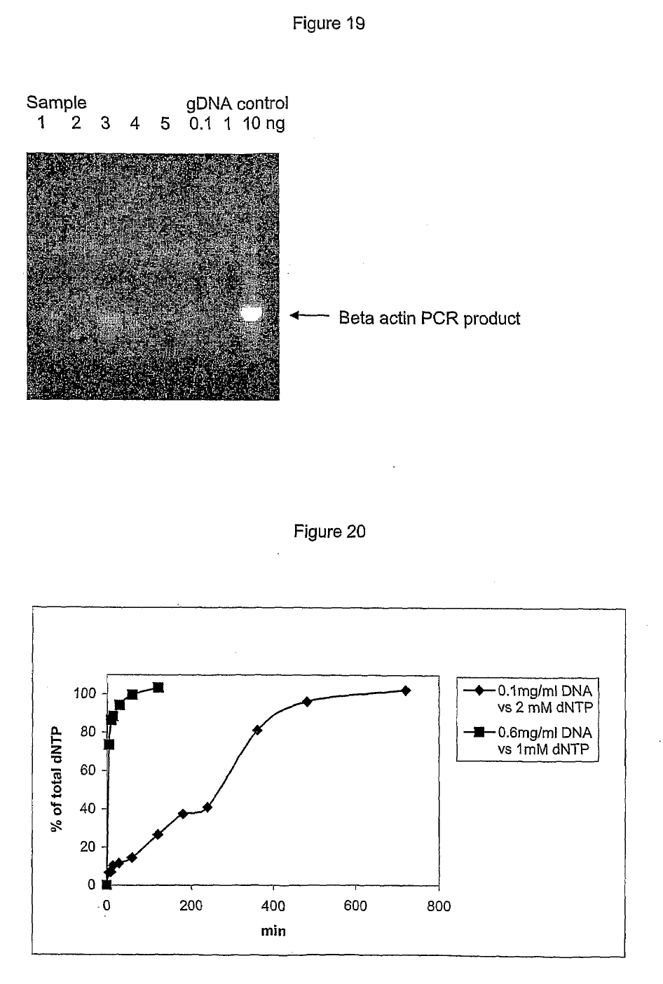

Take 1 out of 20 (0.05 or 0.25 ng original DNA) uL and add to a PCR reaction with Beta-actin primers under standard PCR conditions in 20 uL volumes.
0.1/1/10 ng DNA as controls. The products were run on 1% agarose gel as seen in FIG. 19. The DNA from the amplification reactions could be used to obtain visible bands after the PCR reaction. The control show that more than 1 ng starting material is needed to get visible bands, far more than the starting amount of DNA before amplification with Pol-11.

### Example 20

### Amplification of Genomic DNA for STR Genotyping

Salmon genomic DNA was amplified using the following protocol.

| | | |
|---|---|---|
| DNA | 1/2.5/5 | ng gDNA (salmon) |
| Octamers | 10 | pmol |
| Pol-11 | 0.5 | uL |
| DNTP | 2 | uL |
| 10× Pol buffer | 2 | uL |
| H₂O | to 20 | uL volume |

Heated at 95° C. for 5 min and reannealed at 20° C. for 10 min. Then enzyme was added and the reaction run for 10 cycles at 55° C. for 20 min followed by 20° C. for 2 min. Samples were diluted 1 vs. 5 times in H₂O and used for parental genotyping. The amount of amplified material was measured to be 1 μg starting from either 1 ng, 2.5 ng or 5 ng original amount of DNA. The results are shown in Table 7.

TABLE 7
Original genotyping of the A1 Salmo salar sample.

| | | | | | | | | | |
|---|---|---|---|---|---|---|---|---|---|
| Sample | Marker | Dye | Allele1 | Allele2 | Size 1 | Size 2 | Height 1 | Height 2 | GQ |

Control sample (125 ng genomic DNA per PCR reaction).

| | | | | | | | | | |
|---|---|---|---|---|---|---|---|---|---|
| a1_Ssal11 | Sp2201 | R | 282 | 290 | 282.4 | 290.3 | 423.0 | 376.0 | 0.49 |
| a1_Ssal11 | Sp2210 | B | 134 | 138 | 133.55 | 137.7 | 2576.0 | 2620.0 | 0.49 |
| a1_Ssal11 | Sp2215 | R | 136 | 144 | 135.63 | 144.4 | 10881.0 | 9840.0 | 0.49 |
| a1_Ssal11 | Ssa171 | Y | 239 | | 238.71 | | 2988.0 | | 0.35 |
| a1_Ssal11 | Ssa197 | G | 173 | 189 | 173.17 | 188.96 | 6254.0 | 5303.0 | 0.49 |

Samples amplified with Pol-11 prior to genotyping using different amounts
of starting DNA material (1 ng, 2.5 ng and 5 ng as indicated in Sample column)

| | | | | | | | | | |
|---|---|---|---|---|---|---|---|---|---|
| d1_1 ng | Sp2201 | R | 282 | 290 | 282.8 | 290.7 | 874.0 | 743.0 | 0.39 |
| d1_1 ng | Sp2210 | B | 134 | 138 | 133.51 | 137.77 | 2770.0 | 2928.0 | 0.90 |
| d1_1 ng | Sp2215 | R | 136 | 144 | 135.79 | 144.69 | 3827.0 | 3394.0 | 0.39 |
| d1_1 ng | Ssa171 | Y | 239 | | 238.76 | | 3860.0 | | 0.39 |
| d1_1 ng | Ssa197 | G | 173 | 189 | 173.29 | 189.2 | 1677.0 | 1313.0 | 0.28 |
| d2_2.5 ng | Sp2201 | R | 282 | 290 | 282.46 | 290.58 | 181.0 | 179.0 | 1.0 |
| d2_2.5 ng | Sp2210 | B | 134 | 138 | 133.35 | 137.58 | 1228.0 | 1291.0 | 0.39 |
| d2_2.5 ng | Sp2215 | R | 136 | 144 | 135.54 | 144.53 | 1725.0 | 1864.0 | 0.39 |
| d2_2.5 ng | Ssa171 | Y | 239 | | 238.65 | | 1286.0 | | 0.39 |
| d2_2.5 ng | Ssa197 | G | 173 | 189 | 173.03 | 188.98 | 1053.0 | 775.0 | 0.39 |
| d3_5 ng | Sp2201 | R | 282 | 290 | 282.81 | 290.77 | 141.0 | 156.0 | 1.0 |
| d3_5 ng | Sp2210 | B | 134 | 138 | 133.32 | 137.61 | 1008.0 | 968.0 | 0.39 |
| d3_5 ng | Sp2215 | R | 136 | 144 | 135.77 | 144.52 | 2683.0 | 2964.0 | 1.0 |
| d3_5 ng | Ssa171 | Y | 239 | | 238.73 | | 1361.0 | | 0.39 |
| d3_5 ng | Ssa197 | G | 173 | 189 | 173.03 | 189.01 | 1335.0 | 1028.0 | 0.39 |

Unamplified 5 ng sample. Control sample

| | | | | | | | | | |
|---|---|---|---|---|---|---|---|---|---|
| d4_5 ngUnampl | Sp2201 | R | 282 | 290 | | | | | 0.0 |
| d4_5 ngUnampl | Sp2210 | B | 134 | 138 | 131.25 | 157.95 | 58.0 | 50.0 | 0.01 |
| d4_5 ngUnampl | Sp2215 | R | 136 | 144 | 137.54 | 155.55 | 234.0 | 156.0 | 0.01 |
| d4_5 ngUnampl | Ssa171 | Y | 239 | | | | | | 0.0 |
| d4_5 ngUnampl | Ssa197 | G | 173 | 189 | 183.83 | 199.55 | 66.0 | 81.0 | 0.02 |

Amplified DNA is genotyped correctly for the 5 markers. The positive control sample (a1-Ssal11) contains sufficient amount of DNA for successful genotyping. The negative control sample (d4_{—}5 ngUnampl) using 5 ng of starting DNA material without further amplification fails for all markers. Quality control values (GQ) above 0.25 are considered to indicate reliable results. The results indicate that amounts of DNA, that are to limited for analysis of this type, can be amplified to sufficient amounts that can be successfully used for genotyping and assumingly is correctly amplified.

### Example 21

### Analysis of Sequence and Structure-Function Relationships

The sequences of the polypeptides Pol-11, Pol-3 and Pol-62 were aligned with various other DNA polymerase sequences from public databases using ClustalX software (Thompson et al. 1997). Representative sequences in family A together with sequences of the polypeptides of the invention were selected for final alignment as shown in FIG. 22. Known sequence motifs of family A DNA polymerases were identified in the sequences by visual inspection.

Coordinates of selected crystal structures were analyzed with molecular graphics. The structures were of Taq DNA polymerase (Protein data bank (PDB) ID: 1TAQ), E. coli DNA polymerase Klenow fragment (PDB ID 1D8Y), Bacillus stearothermophilus DNA polymerase fragment (PDB ID 2BDP) and bacteriophage T7 RNA polymerase in complex with nucleic acids (PDB ID 1MSW). The amino acid sequence alignment was partly based on manual adjustment based on the structural superposition of the coordinates of E. coli, Taq and Bst DNA polymerases and published alignments (Blanco et al. 1991, Aliotta et al. 1996; Korolev et al. 1995). The structure of E. coli DNA polymerase was superimposed on the structure of the RNA polymerase using the helices in the fingers domain as reference (Helices O and P) using the program O (Jones et al. 1991). The superimposed structures of the RNA polymerase with associated oligonucleotide template and non-template strands and of the E. coli DNA polymerase, especially the fingers domain, was analyzed in detail with reference to the sequence alignment to visualize location and possible functional importance of residues in the corresponding region in the polypeptides of the invention.

### REFERENCES

Albà, M. M. (2001), Genome Biol 2:reviews3002.1-3002.4.
Aliotta J. M., et al. (1996) Genetic Analysis 12:185-195.
Alsmadi, O. A., et al. (2003), BMC Genomics 4:21-38.
Altschul, et al. (1990), J. Mol. Biol. 215:403-410
Amann, E., et al.: Vectors bearing a hybrid trp-lac-promoter useful for regulated expression of cloned genes in Eshcerichia coli. Gene 25 (1983) 167-178.
Beard, W. A. and Wilson S. H. (2003) Structure 11:489-496.
Blanco, L. and Salas, M. (1996), J. Biol. Chem. 271:8509-8512.
Blanco, L. A., et al. (1989), J. Biol. Chem. 264:8935-8940.
Blanco L., Bernad A, Blasco M A, Salas M. (1991), Gene 100:27-38.
Bowie, et al. (1990) Science 247:1306-1310.
Braithwaite, D. K. and Ito, J.: Compilation, alignment, and phylogenetic relationships of DNA polymerases. Nucleic Acids Res 21 (1993) 787-802.
Brautigam, C. A. and Steitz, T. A. (1998a) J. Mol. Biol. 277:363-377.
Brautigam, C. A. and Steitz, T. A. (1998b), Curr. Opin. Struct. Biol. 8:54-63;
Brock, T. D. and Freeze, H.: Thermus aquaticus gen. n. and sp. n., a nonsporulating extreme thermophile. J Bacteriol 98 (1969) 289-297.
Bruins M. E., et al. Appl Biochem Biotechnol. 2001 February; 90(2):155-86.
Caetano-Anolles, G.: Scanning of nucleic acids by in vitro amplification: new developments and applications. Nat Biotechnol 14 (1996) 1668-74.
Chang, J. R., et al.: Purification and properties of Aquifex aeolicus DNA polymerase expressed in Escherichia coli. FEMS Microbiol Lett 201 (2001) 73-7.
Chien, A., et al.: Deoxyribonucleic acid polymerase from the extreme thermophile Thermus aquaticus. J Bacteriol 127 (1976) 1550-7.
Choi, J. J., et al.: Purification and properties of Thermus filiformis DNA polymerase expressed in Escherichia coli. Biotechnol Appl Biochem 30 (1999) 19-25.
Chung, A. P., et al.: Thermus igniterrae sp. nov. and Thermus antranikianii sp. nov., two new species from Iceland. Int J Syst Evol Microbiol 50 (2000) 209-17.
Dean, F. B., et al. (2001), Genome Res. 11:1095-1099.
Dean, F. B., et al. (2002), Proc. Nat. Acad. Sci. 99:5261-5266.
Degryse, E., et al.: A comparative analysis of extreme thermophilic bacteria belonging to the genus Thermus. Arch Microbiol 117 (1978) 189-196.
Del Solar, G., et al. (1998), Microbiol. Molec. Biol. Rev. 62:434-464.
Detter, J. C., et al. (2002), Genomics 80:691-698.
Fisher, T. S., Darden, T., Prasad, V. R. (2003), J Mol Biol 325:443-459.
Freemont, P. S., et al. (1988) Proc Natl. Acad. Sci. USA 85:8924-8928.
Henne A, et al. (2004) The genome sequence of the extreme thermophile Thermus thermophilus. Nat Biotechnol. 22:547-553.
Hjorleifsdottir, S.: Diversity of thermostable DNA enzymes from Icelandic hot springs, Dept. of Biotechnology. Lund University, Sweden, Lund, 2002.
Hjorleifsdottir, S., et al.: Thermostabilities of DNA ligases and DNA polymerases from four genera of thermophilic eubacteria. Biotechnol Lett 19 (1997) 147-150.
Hjorleifsdottir, S., et al.: Species Composition of Cultivated and Non-Cultivated Bacteria from Short Filaments in an Icelandic Hot Spring at 88° C. Microbial Ecol 42 (2001) 117-125.
Hudson, J. A., et al.: Thermus filiformis sp. nov., a filamentous caldoactive bacterium. Int J Syst Bacteriol 37 (1987) 431-436.
Jones T A, Zou J Y, Cowan S W and Kjeldgaard M, (1991) Acta Cryst. A47, 110-119.
Joyce, C. M. and Steitz, T. A.: Function and structure relationships in DNA polymerases. Annu Rev Biochem 63 (1994) 777-822.
Kristjansson, J. K., et al.: Thermus scotoductans, sp. nov., a pigment producing thermophilic bacterium from hot tap water in Iceland and including Thermus sp x-1. Syst Appl Microbiol 17 (1994) 44-50.
Lage, J. M., et al. (2003), Genome Res. 13:294-307.
Lasken, R. S. and Egholm, M. (2003), Trends Biotechnol. 21:531-535.
Marteinsson, V. T., et al.: Discovery and description of giant submarine smectite cones on the seafloor in Eyjafjordur, northern Iceland, and a novel thermal microbial habitat. Appl Environ Microbiol 67 (2001) 827-33.
Mattila, P., et al.: Isolation and characterization of a new DNA polymerase of Thermus brockianus F500 showing increased thermal stability and fidelity over Taq DNA polymerase, Thermophiles 93: An international conference on the science and technology of thermophiles, Hamilton, New Zealand, 1993.
Needleman, S. B. and Wunsch, C. D. (1970) J. Mol. Biol. 48, 443-453
Nelson, J. R., et al. (2002), BioTechniques 32:S44-S47.
Nielsen et al. 199, Science, 254:1497-1500.
Oshima, T. and Imahori, K.: Description of Thermus thermophilus comb. nov., a nonsporulating thermophilic bacterium from a Japanese thermal spa. Int I Syst Bact 24 (1974) 102-112.
Kiefer J. R., et al. (1997) Structure 15:95-108.
Korolev S., et al. (1995), Proc Natl. Acad. Sci USA 92:9264-9268.
Paez, J. G., et al. (2004), Nucl. Acid. Res. 32:e71.
Perler, F. B., Kumar, S. and Kong, H.: Thermostable DNA polymerases. Adv Protein Chem 48 (1996) 377-435.
Pitulie, C., et al.: Phylogenetic position of the genus Hydrogenobacter. Int J Syst Bacteriol 44 (1994) 620-626.
Pratt, L. A. and Kolter, R.: Genetic analysis of Escherichia coli biofilm formation: roles of flagella, motility, chemotaxis and type I pill. Mol Microbiol 30 (1998) 285-93.
Rice, P. Longden, I. and Bleasby, A. (2000) Trends Genetics 16:276-277.
Rose, T. M., et al.: Consensus-degenerate hybrid oligonucleotide primers for amplification of distantly related sequences. Nucleic Acids Res 26 (1998) 1628-35.
Saiki et al., (1988) Science 239:487-491.
Saitou, N, & Nel, M. (1987), The neighbor-joining method: a new method for reconstructing phylogenetic trees. Mol Biol Evol. 4:406-25.
Skirnisdottir, S.: Phylogenetic characterization of microbial mats and isolation of Thermus spp. and sulfur-oxidizing bacteria from Icelandic hot springs, Dept. of Biotechnology. Lund University, Lund, 2001.
Skirnisdottir, S., et al.: Influence of sulfide and temperature on species composition and community structure of hot spring microbial mats. Appl Environ Microbiol 66 (2000) 2835-41.
Steitz, T. A. and Steitz, J. A. (1993) Proc. Natl. Acad. Sci. USA 90:6498-6502.
Steitz, T. A. (1999), J. Biol. Chem. 274:17395-17398.
Steitz, T. A: and Yin, Y. W. (2004) Philos Trans R Soc Lond B 359:17-23.
Thompson, J. D., et al., (1997) The ClustalX windows interface: flexible strategies for multiple sequence alignment aided by quality analysis tools. Nucleic Acids Research, 24:4876-4882.
Thompson, J. D., et al., (1994). CLUSTAL W: improving the sensitivity of progressive multiple sequence alignment through sequence weighting, positions-specific gap penalties and weight matrix choice. Nucleic Acids Research, 22, 4673-4680
Vieille C, Zeikus G J. Microbiol Mol Bol Rev. 2001 March; 65(1):1-43.
Walker, G. T. et al., “Strand displacement amplification—an isothermal, in vitro DNA amplification technique,” Nucleic Acids Research 20(7): 1691-1696 (1992).
Williams, R. A., et al.: Thermus oshimai sp. nov., isolated from hot springs in Portugal, Iceland, and the Azores, and comment on the concept of a limited geographical distribution of Thermus species. Int J Syst Bacteriol 46 (1996) 403-8.
Williams, et al.: DNA relatedness of Thermus strains, description of Thermus brockianus sp. nov., and proposal to reestablish Thermus thermophilus (Oshima and Imahori). Int J Syst Bacteriol 45 (1995) 495-499.
Wilms, B., et al.: High-cell-density fermentation for production of L-N-carbamoylase using an expression system based on the Escherichia coli rhaBAD promoter. Biotechnol Bioeng 73 (2001) 95-103.
Yin, Y. W, Steitz, T. A. (2004), Cell 116:393-404.

1. An isolated thermostable polypeptide belonging to the DNA polymerase family A which is encoded by a gene sequence obtainable from a Thermus sp. having a non-truncated molecular weight in the range of about 58-68 kDa.

2. The polypeptide of claim 1 having a non-truncated molecular weight in the range of about 61-65 kDa.

3. The polypeptide of claim 1 having a non-truncated molecular weight of about 63 kDa.

4. The polypeptide of claim 1 having DNA polymerase strand-displacement activity.

5. The polypeptide of claim 1 and having proof-reading activity.

6. The polypeptide of claim 4 having DNA polymerase strand-displacement activity which is optimal at a temperature above about 50° C.

7. The polypeptide of claim 6 having substantial DNA polymerase strand displacement activity above about 90° C.

8. The polypeptide of claim 7 having at least about 10% of optimum activity at a temperature of about 90° C.

9. An isolated thermostable polypeptide belonging to the DNA polymerase family A and having DNA polymerase strand-displacement activity, which activity is optimal at a temperature above about 50° C., and having proof-reading activity.

10. The polypeptide of claim 9 having a molecular weight in the range of about 58-68 kDa in non-truncated form.

11. An isolated thermostable polypeptide which belongs to a sub-family sequence-based phylogenetic branch comprising DNA polymerases having the sequences of SEQ ID NO: 4, SEQ ID NO:5 and SEQ ID NO:6, wherein said phylogenetic branch is defined by a phylogenetic tree being prepared with the sequence of said polypeptide and reference sequences shown in FIG. 1 with the use of ClustalX software using the alignment algorithm and the Neighbor Joining Method with default parameters, wherein said branch corresponds to internal branch p stemming from node P in the phylogenetic tree shown in FIG. 1.

12. The isolated thermostable polypeptide of claim 11 which is encoded by a gene sequence obtainable from a Thermus sp.

13. The isolated thermostable polypeptide of claim 11 having a non-truncated molecular weight in the range of about 58-68 kDa.

14. The isolated thermostable polypeptide claim 11 having DNA polymerase strand-displacement activity and having proof-reading activity.

15. The isolated thermostable polypeptide of claim 14 having DNA polymerase strand-displacement activity which is optimal at a temperature above about 50° C.

16. The isolated thermostable polypeptide of claim 1, which polypeptide naturally lacks a 5′-exonuclease domain and comprises a functional 3′ exonuclease domain.

17. The polypeptide of claim 1 comprising the sequence D/E-x-x-R/K-R/K-x-x-x-x-x-x-x-x-R/K (SEQ ID NO: 28) in a region of the polypeptide wherein the left-end residues D/E-x-x-R/K-R/K (SEQ ID NO: 11) align with residues 406-411 of SEQ ID NO: 4, when the sequence of said polypeptide is aligned with the sequence of SEQ ID NO: 4 for optimal alignment.

18. The polypeptide of claim 17 comprising the sequence D/E-x-x-R/K-R/K-Y-x-x-T/S-x-Y-x-x-K/R-I/L-S/T (SEQ ID NO: 29) in said region.

19. The polypeptide of claim 18 comprising the sequence D/E-G-I/L/V-R/K-R/K-Y-A-I/L/V-T/S-x-Y-G-V/L/I-R/K-I/L/V-T/S (SEQ ID NO: 30) in said region.

20. The polypeptide of claim 1 comprising a N-terminal 3′-5′ exonuclease domain having a exonuclease active site sequence motif L-G-V-D-L-E-T-T-G-L-D-P-H (residues 29-41 of SEQ ID NO: 4) in a region of the polypeptide wherein the left-end residues L-G-V-D align with residues 29-32 of SEQ ID NO: 4, when the sequence of said polypeptide is aligned with the sequence of SEQ ID NO: 4 for optimal alignment.

21. The polypeptide of claim 1 comprising a C-terminal polymerase domain having a polymerase active site sequence motif L-K-A-D-F-S-Q-I-E-L-R-J-A-A-A in a region of the polypeptide wherein the residues align with residues 337-351 of SEQ ID NO: 4, when the sequence of said polypeptide is aligned with the sequence of SEQ ID NO: 4 for optimal alignment.

22. The polypeptide of claim 1 having a specific activity of at least 10.000 Units/mg when assayed with a DNA polymerase assay at 55° C. in TEG buffer (25 mM Tris-hydrochloride, pH 8; 50 mM disodium EDTA; 1% glucose), deoxyribonucleoside triphosphates (250 μM each mixed with 2 μCi of [methyl-3H] Thymidine 5′-triphosphate); 30 μg of activated DNA; and 0.02-0.06 μg of the DNA polymerase enzyme, in a 50 microliter reaction and assayed from 1-20 minutes; where one Unit of enzyme activity is defined as the amount which catalyzes the incorporation of 10 nmol of total nucleotides into acid-insoluble product under said conditions after 30 min.

23. The polypeptide of claim 22 having a specific activity of at least 100.000 Units/mg.

24. An isolated polypeptide selected from:
a. a polypeptide comprising the sequence of SEQ ID NO: 4;
b. a polypeptide comprising the sequence of SEQ ID NO: 5;
c. a polypeptide comprising the sequence of SEQ ID NO: 6;
d. a polypeptide having at least 40% sequence identity to any of the sequences of SEQ ID NO: 4, SEQ ID NO: 5 or SEQ ID NO: 6 and having substantial DNA polymerase strand displacement activity and proof-reading activity.

25. The polypeptide of claim 23 having at least 60% sequence identity to any of the sequences of SEQ ID NO: 4, SEQ ID NO: 5 or SEQ ID NO: 6 and having substantial DNA polymerase strand displacement activity and proof-reading activity.

26. The polypeptide of claim 24 having at least 75% sequence identity to any of the sequences of SEQ ID NO: 4, SEQ ID NO: 5 or SEQ ID NO: 6 and having substantial DNA polymerase strand displacement activity and proof-reading activity.

27. An isolated polynucleotide selected from the group consisting of:
a. a polynucleotide comprising the sequence of SEQ ID NO: 1;
b. a polynucleotide encoding the polypeptide of SEQ ID NO: 4;
c. a polynucleotide comprising the sequence of SEQ ID NO: 2;
d. a polynucleotide encoding the polypeptide of SEQ ID NO: 5;
e. a polynucleotide comprising the sequence of SEQ ID NO: 3;
f. a polynucleotide encoding the polypeptide of SEQ ID NO: 6;
g. a polynucleotide encoding a polypeptide which polypeptide has DNA polymerase strand displacement activity above about 55° C. and has at least 40% sequence identity to any of the amino acid sequences of SEQ ID NO: 4, SEQ ID NO: 5 and SEQ ID NO: 6;
h. a polynucleotide that hybridizes under stringent conditions to the complement of any of the nucleotide sequences of SEQ ID NO: 1, SEQ ID NO: 2, SEQ ID NO: 3; and
i. a polynucleotide that is complementary to any one of the above defined polynucleotides of a-h).

28. The polynucleotide of claim 27 encoding a polypeptide which polypeptide has DNA polymerase strand displacement activity above about 55° C., has proof-reading activity and has at least 60% sequence identity to any of the amino acid sequences of SEQ ID NO: 4, SEQ ID NO: 5 and SEQ ID NO: 6.

29. An isolated polynucleotide encoding a polypeptide of claim 1.

30. A DNA construct comprising an isolated nucleic acid molecule of claim 27, operatively linked to a regulatory sequence.

31. A host cell comprising a DNA construct of claim 30.

32. A method of amplifying a target nucleic acid sequence, the method comprising,
bringing into contact a DNA polymerase of as defined in claim 1, and a target sample and optionally a set of primers, and incubating the target sample under conditions that promote replication of the target sequence,
wherein replication of the target sequence results in replicated strands, wherein during replication at least one of the replicated strands is displaced from the target sequence by strand displacement replication of another replicated strand.

33. The isolated thermostable polypeptide of claim 9, which polypeptide naturally lacks a 5′-exonuclease domain and comprises a functional 3′ exonuclease domain.

34. The isolated thermostable polypeptide of claim 11, which polypeptide naturally lacks a 5′-exonuclease domain and comprises a functional 3′ exonuclease domain.

35. An isolated polynucleotide encoding a polypeptide of claim 9.
